(19) United States
(12) Patent Application Publication
(10) Pub. No.: US 2018/0364188 A1
(43) Pub. Date: Dec. 20, 2018

(54) MODULAR CHEMIRESISTIVE SENSOR FOR IN VITRO DIAGNOSTIC AND GAS SENSING APPLICATIONS

(71) Applicant: INNOSENSE LLC, Torrance, CA (US)

(72) Inventors: Anamika Ray, Torrance, CA (US); Yifan Tang, Torrance, CA (US); Maksudul M. Alam, Glendora, CA (US); Jeffrey Paul, Torrance, CA (US); Corey Selman, West Hills, CA (US); Uma Sampathkumaran, Torrance, CA (US)

(21) Appl. No.: 15/989,125

(22) Filed: May 24, 2018

Related U.S. Application Data
(63) Continuation-in-part of application No. 15/851,587, filed on Dec. 21, 2017.
Application No. 15/851,587 is a continuation-in-part of application No. 14/658,034, filed on Mar. 13, 2015, now Pat. No. 9,896,772.
(60) Provisional application No. 62/536,940, filed on Jul. 25, 2017.
(60) Provisional application No. 61/952,557, filed on Mar. 13, 2014.

(51) Int. Cl. G01N 27/12; C25D 11/00; C25B 3/00; H01L 51/00; G01N 33/487

(52) U.S. Cl. CPC G01N 27/127; C25D 11/00; C25B 3/00; G01N 2800/7028; H01L 51/0006; G01N 33/48707; G01N 2800/26; H01L 51/0049

(57) ABSTRACT

A sensor array comprising multiple discrete sensors for providing detection and prognosis of various diseases. The array is made up of multiple discrete sensors, each of which has a first and a second noble metal electrode on a silicon substrate, said electrodes separated by a gap. An electrically conductive pathway across the gap between the first and second noble electrodes is provided by a nano-network of functionalized polymer nanowires or carbon nanotubes (SWNTs) the arrangement providing a sensor. The multiple discrete sensors comprise a reference cell and multiple detection sensors functionalized using a panel of capture molecules to detect the same or different diseases or biological functions.

This application claiming priority based on U.S. Application No. 62/536,940 filed Jul. 25, 2017 and is a Continuation in Part of U.S. patent application Ser. No. 15/851,587 filed Dec. 21, 2017 which is a continuation in part of U.S. patent application Ser. No. 14/658,034, filed Mar. 13, 2015 which claims priority based on U.S. Patent Application No. 61/952,557, filed Mar. 13, 2014, which is incorporated herein in its entirety. This invention was made with government support under (1) Grant: DE-SC0008210—awarded by Department of Energy, Chicago, Ill., (2) Grant: 5R43AG029006, awarded by National Institutes of Health, Washington, D.C., (3) Grant: 1R43ES023707-01 awarded by National Institutes of Health, Washington, D.C., (4) Grant:1R43 A1126931-01A1 awarded by National Institutes of Health, Washington, D.C., and (5) W81XWH-17-C-0188 awarded by Defense Health Agency Contract: HQ0147-13-C-7333—awarded by Missile Defense. Agency (MDA), Redstone Arsenal, AL.

### FIELD

The present invention relates to a modular chemi-resistive sensor incorporating conductive polymeric nanowires or carbon nanotubes and devices using and/or incorporating these sensors. In particular, these devices include, but are not limited to modular chemiresistive sensors for hypergolic fuel and oxidizer leak detection, carbon dioxide monitoring and detection of specific disease biomarkers. Various embodiments include biosensor platforms comprising microelectronic devices which use conductive polymeric fibers or single walled carbon nanotubes (SWNT) as the active sensing materials. The fibers or carbon nanotubes can then be further functionalized with biomarkers, such as antibodies that detect specific antigens or engineered DNA, RNA, aptamers, or pengineered miRNAs and their variants that detect corresponding complementary engineered and recombinant molecules or structures either individually or in a combination mixture. Multiple devices with different biomarkers can be used in tandem to provide a diagnosis matrix for more precise and accurate results on targeted disease or biological process monitoring.

### BACKGROUND

Missile systems, such as the Theatre High Altitude Area Defense (THAAD) ballistic missiles, use hypergolic fuels and oxidizers as a means of propulsion. These hypergolic propellants and oxidizers are corrosive, carcinogenic, toxic, and present fire hazards when a leak is present. Their storage and deployment are thus crucial to ensure personnel safety and mission success. The hypergolic fuel used in missiles is hydrazine or monomethyl hydrazine (MMH), and the oxidizer used in missiles is mixed oxides of nitrogen (MON-25) that is a mixture of dinitrogen tetroxide (N₂O₄), nitrogen dioxide (NO₂) and nitric oxide (NO). N₂O₄ is a dimer of NO₂. Under equilibrium conditions, nitrogen tetroxide (NTO) exists as a mixture of N₂O₄ and NO₂. Therefore, detection of MMH and NO₂ exterior of their storage vessels would indicate a leak in the system.

Electrochemical, chemiluminescence, chemical resistance, absorption, and fluorescence-based detection systems have been developed for the detection of hypergolic fuel and oxidizer leaks. However, these leak-detecting sensor devices suffer from drawbacks such as lack of specificity, less effective operation at elevated temperatures, and cell leakage problems leading to maintenance challenges. In addition, the prior art electrochemical monitoring devices can operate in the range −20° C. to +71° C. However, the response time of prior art electrochemical systems at −20° C. is typically 55 minutes at 100 ppm for NO₂, and their sensitivity is typically about 100 ppm for both MMH and NO₂. Prior art systems also should be replaced annually—which is a maintenance burden and drives system lifecycle costs. Thus, the development of a highly reliable and accurate transducer element to detect rapid changes in concentration of hypergolic fuels and oxidizers within a tactical leak detection subsystem is desired.

Atmospheric levels of carbon dioxide (CO₂) have risen significantly from pre-industrial levels of 280 ppm to present levels of 404 ppm. Predictions on future energy use indicate a continued increase of atmospheric CO₂ unless major changes are made in the way energy is produced and how carbon is managed. Due to current concerns about global climate change related to increased CO₂ emissions, efforts are underway to better utilize both terrestrial and geologic CO₂ sinks as forms of carbon management, offsetting emissions from fossil fuel combustion and other human activities. The storage of industrially generated CO₂ in deep geologic formations is considered a viable method and important for reducing CO₂ (green house emissions) from the atmosphere. Roughly a billion metric tons of CO₂ has to be sequestered annually to make an impact. The Department of Energy (DOE) carbon sequestration “Monitoring Verification and Accounting (MVA)” program requires sensors to monitor, measure and account for 99% of CO₂ in the injection zones to confirm safe and permanent storage of CO₂ in geologic formations, especially in the near-surface and subsurface environments over a large area with improved accuracy and long-term durability. Reliable and cost-effective monitoring systems are critical to safe permanent storage. Light Detection and Ranging (LIDAR) or satellite-based technologies are only effective for atmospheric or above ground CO₂ monitoring. By the time leaked CO₂ appears above the surface, significant damage may have occurred to ground water and the surrounding ecosystem. Therefore, a reliable and cost-effective near-surface/subsurface CO₂ monitoring system is critical to confirming the safe and permanent storage of 99% of CO₂ in the geologic injection zones.

Alzheimer's disease (AD) is the most common form of dementia. AD and other forms of dementia impose a tremendous financial burden on the health care system and the general economy. According to the Alzheimer's Association, the cost of caring for AD patients is estimated to be $203 billion in the United States in 2013. In addition, 15.4 million Americans provide unpaid care valued at $216 billion for persons with AD and other dementias. Unless addressed, the cost of AD is estimated to reach $1.2 trillion by 2050. Therapeutics can delay the onset of AD to an extent; however, their efficacy depends on early diagnosis. In 2012, the U.S. Food and Drug Administration (FDA) approved Amyvid™, a radiopharmaceutical imaging agent for positron emission tomography (PET) scans that measure the brain β-amyloid plaque density in-vivo in patients. The PET scans are highly sensitive. However, Amyvid PET scan is not a test for predicting the development of AD-associated dementia and is not intended to monitor patient responses to AD therapy. Amyvid does not replace other diagnostic tests used in the evaluation of cognitive impairment. In addition, PET scans are costly, time consuming, require skilled personnel, and cannot be used as a point-of-care application in doctor's offices and clinics. Another diagnostic method based on a flow cytometric test of Aβ phagocytosis for the detection of AD biomarkers in blood was reported. Neither of these approaches can easily be converted into a cost-effective diagnostic or research tool. Despite the utmost importance, no cost-effective biosensor technologies have been marketed to detect AD biomarkers. Therefore, there is an urgent need to develop technologies for AD screening and early presymptomatic diagnosis. Developing a simple and low-cost biosensor for reliable early diagnosis of AD in point of care facilities is needed.

Cancer is a group of diseases characterized by uncontrolled growth and spread of abnormal cells. It is the leading cause of death worldwide. The United States National Cancer Institute Society has estimated that there are 1,444,920 new cases of cancer and about 559,650 deaths in the United States each year—more than 1500 deaths per day. The National Institutes of Health estimates that the overall costs for cancer in each year are $206.3 billion: $78.2 billion for direct medical costs; $17.9 billion for indirect morbidity costs; and $110.2 billion for indirect mortality costs. This problem underscores the need for reliable and cost-effective methods for early detection and diagnosis of cancer. A device to monitor cancer therapy progress is also needed. There are several different kinds of cancer. For example: (i) Prostate cancer (PC) is the most common type of cancer found in American men. The American National Cancer Society estimates that there are 218,890 new cases of PC and 27,050 deaths in the United States in each year. PC is the second leading cause of cancer death in men in the United States. Prostate specific antigen (PSA) is the over-expressed biomarker of PC, and is crucial for the detection and diagnosis of PC. (ii) Breast cancer (BC) is the most frequently diagnosed cancer in women. The American National Cancer Society estimates that there will be about 240,510 new cases of breast cancer among women and, as estimated, 40,910 breast cancer deaths (40,460 women and 450 men) are expected in the United States each year. BC ranks second among cancer deaths in women. A protein called human epidermal growth factor receptor 2 (HER-2/neu) is overexpressed in about 20-30% of BCs, which tend to be more aggressive. This overexpressed HER-2/neu protein is an important therapeutic target/biomarker for diagnosis and prognosis of BC. (iii) Lung cancer (LC) accounts for the most cancer related deaths in both men and women. An estimated 213,380 new cases and 160,390 deaths, accounting for about 29% of all cancer deaths, are expected to occur in the United States in each year. Epithelial cell adhesion molecule (EpCAM) protein is an important biomarker of LC. A primary cause of poor survival rates is that many cancers are detected late, after they have spread or metastasized to distant sites. For most types of cancer, the earlier the detection the greater the chances of survival. Therefore, there is an urgent need for devices or methods that can accurately and reproducibly measure multiple cancer biomarkers or circulating tumor cells in bodily fluids or other specimens obtained by minimally invasive methods.

### SUMMARY

The present invention relates to a modular chemiresistive sensor. In particular, a modular chemiresistive sensor for detecting leaks of stored chemicals, particularly hypergolic fuel and oxidizer leak detection, carbon dioxide monitoring and detection of disease biomarkers. The sensor preferably has two gold or platinum electrodes mounted on a silicon substrate where the electrodes are connected to a power source and are separated by a gap of 0.5 to 4.0 μM. A polymer nanowire or carbon nanotube may be functionalized with receptor complexes for chemical sensors or a panel of biomarkers for disease detection. The functionalized nanowires/nanotubes span the gap between the electrodes providing an electrical connection between the electrodes. The electrodes are further connected to a circuit board having a processor and data storage capabilities, where the processor can measure current and voltage values between the electrodes and compare the current and voltage values with current and voltage values stored in the data storage and assigned to particular concentrations of a pre-determined substance, or a variety of other substances, to be detected and concentrations thereof detected.

### BRIEF DESCRIPTION OF THE DRAWINGS

The present invention described herein will become apparent from the following detailed description considered in connection with the accompanying drawings, which disclose several embodiments of the invention. It should be understood, however, that the drawings are designed for the purpose of illustration and not as limits of the invention.

FIG. 1A is a photograph of a preferred lab set up to perform electro-polymerization.

FIG. 1B is a photograph of a small-volume electrochemical cell with electrodes dipped in monomer solution.

FIG. 2 shows an exploded view and an assembled view of a preferred embodiment of a polymer nanowire microelectronic detector (PNMD) and sensor housing.

FIG. 2A is a schematic of a preferred embodiment of a circuit diagram for the circuit board assembly in FIG. 2.

FIG. 2B is a second schematic of a preferred embodiment of a layout of the circuit board assembly in FIG. 2.

FIG. 3A is a schematic of a preferred embodiment of a PNMD sensor.

FIG. 3B is a schematic of the preferred embodiment of a PNMD sensor with a conductive material bridging the gap between electrodes.

FIG. 4 are chemical structures of electroactive aniline monomer (left) and amine functionalized 2-(2-aminoethyl) aniline (right) for generating conducting polymer nanowires for sensing MMH and NO₂.

FIG. 5 is a diagram representing a chemical reaction that decreases in conductivity of polyaniline as a result of the reducing nature of MMH.

FIG. 6 is a diagram showing a change in chemical structure and an increase in the conductivity of alkylamine functionalized polyaniline as a result of a reaction with NO₂, a strong oxidizing agent.

FIG. 7 is a diagram showing chemical structures of other conducting polymers that can be used for the formation of PNMD as an MMH and NO₂ sensor.

FIG. 8A is a graph of current voltage (I-V) curve of a sensor device before and after nanowire growth.

FIGS. 8B-D are SEM images of nanowires grown across a 2 μm gap between electrodes in a sensor.

FIG. 9A is a schematic of an embodiment of a portable handheld bio-sensor for point of care early diagnosis of disease.

FIG. 9B is a schematic of an embodiment of a sensor chip array used in FIG. 9A and FIG. 9C is an enlarged single biochip used in the array of FIG. 9B.

FIG. 10 is a schematic diagram of a test setup for gas sensing to measure I-V curves of a nanowire sensor.

FIG. 11A is a graph of the response of a PNMD sensor to increasing NO₂ concentrations at room temperature. The arrow in FIG. 11A indicates the direction of change in signal.

FIG. 11B is a graph of sensor response as a function of NO₂ concentration up to 100 ppm. The error bards (where visible) indicate ±5% of the value. R² indicates the increasing value for the linear trend line.

FIG. 12A is a graph of I-V curves of the PNMD sensor with increased MMH concentrations at 23° C. The arrow indicates the direction of change in signal. FIG. 12B shows change in sensor response as a function of MMH concentration up to 300 ppm. The error bars (where visible) indicate ±5% of the value. R² indicates the regression value for the linear trend line.

FIG. 13A shows a signal response versus time (V-t) plot for NO₂ sensing by a PNMD sensor tested with a breadboard device. The flat line at 1.5V indicates the base curve of the sensors when exposed to an N₂ (gas stream) alone. The other line graphs the sensor response to various concentrations of NO₂ (0 to 100 ppm) at 40° C.

FIG. 13B shows the percent change (triplicate measurements) in sensor response as a function of NO₂ concentration. The error bars (where visible) indicate ±5% of the value. R₂ indicates the regression value for the linear trend line.

FIG. 14A is a graph showing signal response versus time (V-t) plot for MMH sensing by PNMD sensor tested with a breadboard device. The flat line at 1.5V indicates the base curve of the sensors when exposed to N₂ in air alone.

FIG. 14B is a graph indicating the sensor response to various concentrations of MMH (0 to 300 ppm) at 40° C. The percent change (triplicate measurements) in sensor response as a function of MMH concentration. The error bars (where visible) indicate ±5% of the value. R² indicates the regression value for the linear trend line.

FIG. 15A is a graph of PNMD performance detecting NO₂; FIG. 15B is a graph of showing response to MMH concentrations. These graphs emphasize the responses of NO₂ and MMH compared to the interfering gases at 0-100 ppm NO₂ (FIG. 15A) and 0-300 ppm MMH (FIG. 15B) response plotted with the response from the interfering gases.

FIG. 16A and FIG. 16B show the response graph as a function of CO₂ concentrations up to 10,000 ppm for two different nanowire sensors. Each point is the average of three measurements, and the error bars (where visible) indicate ±5% of the value. R₂ indicates the regression value for the trend line.

FIG. 17 is a graph of I-V curves of an anti-Aβ42-modified nanowire device exposed to PBS solutions in a solution with various Aβ42 concentrations.

FIG. 18 is a graph of current versus time plots of an anti-PSA-antibody attached nanowire biosensor exposed to PSA antigen with 0, 10 and 14 ng/ml;

FIG. 19 shows the chemical structure and formation of functionalized biocompatible polypyrrole and FIG. 19A shows the functionalized polypyrrole for biosensor use.

FIG. 20 is a diagram showing conjugation of antibody to functionalized biocompatible polypyrrole.

FIG. 21 is a schematic diagram showing a testing device with enlarged views illustrating a polymer nanowire assembly (FIG. 21A), a PSA-AB antibody attached (FIG. 21B) and the interaction of the antibody with corresponding disease biomarker antigen or protein for detecting disease (FIG. 21C).

FIG. 22 is a diagram showing a reversible interaction of CO₂ with alkylamine (R—H₂) conducting polymers to form a carbonate (R—NHCO2).

FIG. 23A and FIG. 23B illustrate[[s]] efficient growth of polymer nanowires with a porous nano-network from 0.2 M aniline monomer in 0.4 M HNO₃ electrolyte solution where FIG. 23A is a graph showing an electrochemical voltage-time growth profile and FIG. 23B is a SEM image of the polymer nanowires formed.

FIGS. 24A and 24B are graphs showing the responses of polymer nanowire sensor devices fabricated from 0.2 M aniline in 0.4 M HNO₃ where FIG. 24A illustrates 10, 20, 50, 60, 80 and 100 ppm NO₂, and FIG. 24B illustrates 10, 50, 100, 200 and 300 ppm MMH.

FIG. 25 is an SEM image of CNTs incorporated within the 2 μm gap between microelectrodes in a sensor device.

FIG. 26 is a histogram showing the distribution of CNT bundle diameters deposited within the 2 μm gap between microelectrodes.

FIG. 27A shows calibration curves and FIG. 27B shows and a bar graph of sensor devices for Hg²⁺, Mn²⁺, Mg²⁺, Ca²⁺, and control (p<0.01 by t-test).

FIG. 28A shows I-V curves and FIG. 28B shows a calibration curve of a representative mercury sensor device including a control when exposed to varying concentrations of Hg²⁺ in water.

FIG. 29A is I-V curves from a representative biosensor showing the Aβ1-42 Ab-Ag response.

FIG. 29B is I-V curves from a control device to which no antibodies were attached exposed to same concentration of Ags in PBS as in FIG. 29A.

FIG. 29C is derivative curves showing the overall response of a biosensor to varying concentrations of Aβ 1-42 Ag in PBS.

FIG. 29D shows a derivative curve response of a control device with no Abs attached exposed to the same concentration of Ags. p=0.002 (t test) as in FIG. 29C.

FIG. 30A is a line graph and FIG. 30B is a bar chart showing the overall response of multiple biosensor devices to varying concentrations of Aβ 1-42 Ag in PBS wherein the lower curve in FIG. 30A represents the response profile of the Ab-conjugated devices to its corresponding Ags and the upper line represents the response of control devices (with no antibody conjugation) exposed to the same dilution series of Ags.

FIG. 31 is a bar chart summarizing the response profile of biosensor devices conjugated with AD-specific Abs and exposed to corresponding Ags in PBS.

FIG. 32A is top perspective view of a microelectrode device (referred to as a MED) embodiment incorporating features of the invention described herein.

FIG. 32B is a side view of the embodiment of FIG. 32A.

FIG. 32C is top perspective view of a microelectrode device embodiment.

FIG. 33A is a side perspective view of the assembled device holder with the device in place.

FIG. 33B is a top view of an assembled device holder with aluminum foil electrical contacts.

FIG. 34 is another SEM image showing the SWNTs bridging the 2 μm gap between the electrodes.

FIG. 35 is a schematic diagram of the step-by-step fabrication: of a multiple sensor array where step (a) shows a patterned microelectrode device array, step (b) shows growth of SWNTs on the electrode junctions, step (c) shows the device of step (b) including surface passivation, step (d) shows custom designed site-specific sampling wells to facilitate specific conjugation of probes (antibodies-Abs) and step (e) shows devices with Abs antibodies grafted thereon.

FIG. 36 shows a multiple device testing setup with spring loaded pin contacts.

FIG. 37A is an I-V curve from one representative device showing the cortisol Ab-Ag response; FIG. 37B is an I-V curve from a control device to which no Abs antibodies were attached, the device exposed to same concentration of antigens (Ags) in PBS.

FIG. 38A is a bar graph showing the average response of multiple devices to varying concentrations of pooled cortisol compared to controls. The longer bars represent the response profile of the anti-cortisol Ab-conjugated devices to their corresponding (high) pooled salivary cortisol while the shorter bars represent the response of control devices to low pooled salivary cortisol exposed to the same dilution series of Ags. The X-axis is the concentrations of Ags in increasing order of exposure.

FIG. 38B is a calibration curve showing the average sensor response change as a function of increasing cortisol Ag. R² indicates the regression value for the linear trend line.

FIG. 39A, a bar graph derived from the calibration curve, summarizes the average negative response trend of multiple devices to varying concentrations of C-reactive protein (CRP) when compared to controls. The long bars represent the response profile of the anti-CRP Ab-conjugated devices to its corresponding salivary CRP while the shorter (blue) bars represent the response of control devices exposed to the same dilution series.

FIG. 39B is a calibration curve showing the average sensor response change as a function of increasing CRP Ag. R² indicates the regression value for the trend line.

FIG. 40A is a three axis graph showing the average overall response of devices to varying concentrations of human salivary cortisol compared to controls (low salivary cortisols and cross reactivity with CRP Ags). The longer bars represent high cortisol, the shorter bars in the front represent low cortisol and the shortest bars in the rear show cross-reactivity.

FIG. 40B is a three axis graph showing the average overall response of devices to varying concentrations of human salivary CRP compared to controls (low salivary CRP and cross reactivity with cortisol Ags). The longer bars represent high cortisol, the shorter bars in the front represent low cortisol and the shortest bars to the rear show cross-reactivity.

FIG. 41 is a simplified schematic diagram of a modified circuit board for testing a prototype system.

FIG. 42 is an embodiment of a USB-C detector testing module incorporating features of the invention showing a hand held reader with the stick assembly extending from the top, the stick assembly containing a sensor.

FIG. 43A is an assembled view of the stick assembly.

FIG. 43B is an exploded view of the stick assembly of FIG. 43A including the disposable sensor chip.

FIG. 44A shows the front surface and FIG. 44B shows the rear surface of the sensor in the chip including a board assembly showing six (6) sensor chips.

### DETAILED DESCRIPTION

Various embodiments are described with reference to the drawings, wherein like reference numerals are used to refer to like elements throughout. In the following description, for purposes of explanation, numerous specific details are set forth in order to provide a thorough understanding of one or more embodiments. However, such embodiment(s) may be practiced without these specific details.

In the following paragraphs, the present invention will be described in detail by way of example with reference to the attached drawings. Throughout this description, the preferred embodiments and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

As used herein PNMD will be used to refer to a polymer nanowire or carbon nanotube microelectronic leak detector that uses an innovative sensing technology for sensitive and accurate detection gases, particularly MMH and NO₂ under dry nitrogen. The term PNMD will generally be used herein to refer to embodiments of a sensor, though not always for detection of hypergolic leaks. As will be shown, the sensors can be configured to detect other substances.

Miniaturized and low-power consuming PNMDs are fabricated by direct and site-specific growth of polymer nanowires (or deposition of carbon nanotubes) at patterned microchannel electrode junctions. The nanowires are preferably grown from electroactive aniline and functionalized aniline monomers (FIG. 4) using a template-free electrochemical method to form porous, electrically conductive filaments.

Advantages of a one step fabrication of SWNT biosensors for use in microelectrode devices, and subsequently for biosensor arrays provides a robust, highly sensitive assay with an ability to withstand chemical and mechanical shock. On the other hand, biomarkers, such as antigen/antibodies and/or engineered DNA/RNA/aptamers/engineered oligos/engineered miRNAs and their complex structures are found to be closely related to (i.e., signal the presence of) various diseases and/or biological processes and thus are important for clinical utility such as diagnosis or monitoring. However, the current bio-chemical, immunological or spectroscopy based processes to detect the presence of those biomarkers have multiple draw backs such as selectivity, accuracy, testing speed, processing of samples and cost.

By utilizing the advantages of the SWNT sensors in combination with their ability to detect biomarkers, the instruments and methods described herein provide precise and reproducible in situ sensing of a disease state. Miniaturized sensor platforms feature fast response times, low power needs and the likelihood of low-cost mass production and easy scale-up.

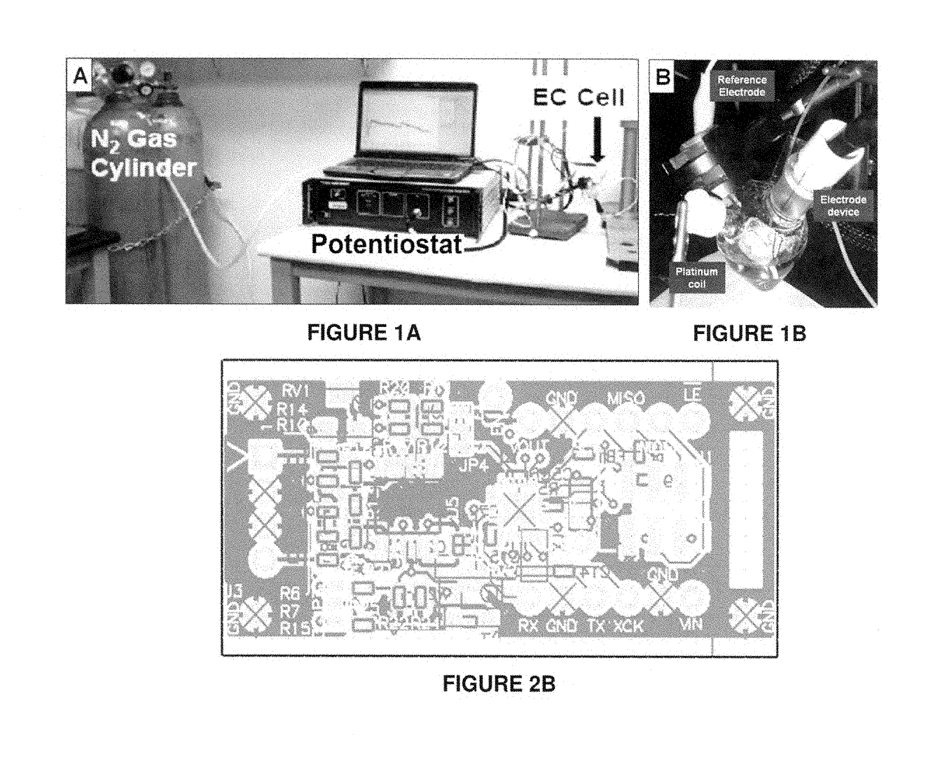

Referring now to FIG. 1, a preferred lab set up for direct electrochemical growth of polymer nanowires in a gap between two electrodes is shown. Preferably, an aqueous solution of an electroactive monomer in 1.0M nitric acid, perchloric acid or hydrochloric acid is used to generate the conducting polymer nanowires. A concentration of a monomer is preferably varied from 0.1M to 0.5M to generate different densities of nanowires. A small volume flask is filled with approximately 16 ml of monomer solution in which a wire-bonded electrode junction device is submerged. One side of the device acts as the working electrode. For a counter electrode a platinum coil is used. The platinum coil preferably has 10-12 turns and a wire diameter of 0.25 mm. A silver/silver chloride reference electrode is preferably used to monitor the reaction voltage. The solution is preferably purged with nitrogen for ten minutes prior to starting the electrochemical reaction. Nitrogen is constantly flowed into the flask during the process to maintain a neutral and non-oxidative environment above the solution. An oxidative potential is applied to one side of the electrode junction device, and the platinum coil is grounded. A potentiostat system (e.g. Princeton Applied Research model 263A-1 potentiostat/galvanostat) is preferably used to provide the potential difference. This method oxidizes the monomers and triggers a chain reaction resulting in the formation of polymer nanowires.

A variety of substances can be used for, or in place of, nanowires including carbon nanotubes, graphene nanofilms, silicon nanowires, tin nanowires, titanium nanowires, metal oxids (zinc, magnesium, calcium, manganese, titanium, tin, and copper oxide) nanowires and nanotubes, graphene, and quantum dots. These various substances can then be used for chemiresistive microelectronic sensing applications.

The PNMDs' sensitivity to both MMH and NO₂ was tested. The PNMDs were tested for stability, sensitivity, response time, and temperature dependence from −46° C. to +71° C. PNMDs exhibit the ability to detect and distinguish 10-300 ppm of MMH and 10-100 ppm of MON-25 within 10 minutes. PNMDs are resistant to interfering gases such as oxygen, carbon dioxide, methane, acetone, alcohol (methanol), and water, with only a slight sensitivity to ammonia. PNMDs show promising stability to shock, vibration and long-term testing. A small footprint PNMD with electronic circuitry preferably provides calibration-free operation, eliminating drift and the effects of temperature and humidity. PNMD is suitable for integration with missiles, highly reliable detection of MMH and NO₂, an effective early warning system for trace detection of hypergolic fuel leaks with resistance to trace interferents, vibration and mechanical perturbations. PNMDs are also suitable to operate under a wide range of temperatures and environments.

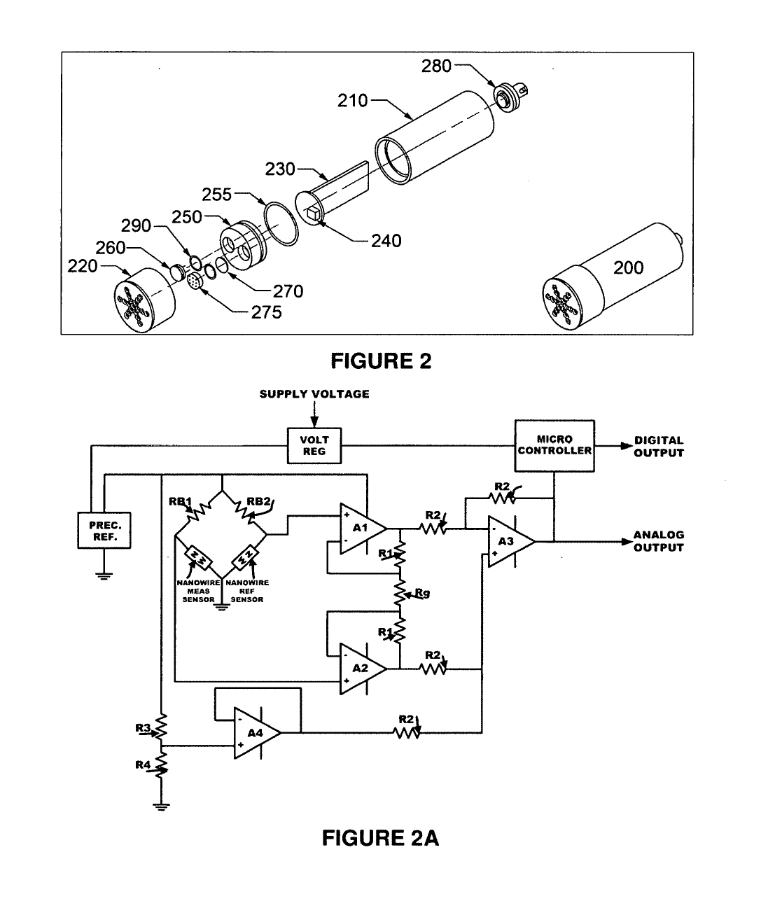

Referring to FIG. 2, an exploded view of a preferred embodiment of the sensor assembly 200 for the PNMD is shown. The sensor assembly preferably has a main housing 210 and a protective cap 220 to contain a circuit board assembly 230 with a PNMD sensor (or array of sensors) 240 mounted thereon. A sensor insert plug 250 and O-ring 255 preferably separate the PNMD sensor 240 and circuit board assembly 230 from a temperature sensor 260. The sensor insert plug 250 preferably comprises a member 270 and membrane retainer 275. A MIL standard connector 280 is mounted to housing 210 to connect the sensor assembly 200 to data and/or power sources (not shown).

FIGS. 2A and 2B show schematics of the preferred embodiment for the circuit board assembly 230. In FIG. 2A, the circuit 230 used for the nanowire sensors has a balanced bridge design with one sensor for reference and a second one for measurement. The bridge is formed by two nanowire sensors 240 and two precision resistors (RB1 and RB2). These fixed resistors are by design nearly equal to the room temperature resistance of the nanowire sensors. A precision 3-volt reference source drives the bridge. This voltage source is powered by a voltage regulator to minimize the dependency on the supply voltage. A differential voltage is formed at the two nodes of the bridge, which are connected to an instrumentation amplifier formed by amplifiers A1, A2 and A3. The gain of the instrumentation amplifier is controlled by a single resistor, Rg, and is equal to G=1±(2R1/Rg). Initially, the gain (G) of this circuit is set to 1 due to the high sensitivity of the nanowire sensors to the presence of the gas.

Although resistors RB1 and RB2 are selected to balance the bridge as well as possible, there is inevitably some small residual differential voltage. The nominal output of the instrumentation amplifier can be set by adjusting the offset input that is supplied by amplifier A4. This is a unity-gain buffer amplifier that sets the offset voltage based upon the resistive divider formed by R3 and R4. This resistive divider is driven by the precision reference source so that the offset will track any small changes in the reference source as temperature is varied. The final output of the instrumentation amplifier is provided as an analog output for data logging purposes. It is also provided to the input of an ADC within the on-board microcontroller, where it can be digitized, processed, and sent out through a serial communication port. The entire circuit is designed on a circuit board 230 that preferably measures 0.9 in. ×1.7 in., which includes space for some connectors in order to make the testing more convenient. FIG. 2B shows the layout of main circuit board.

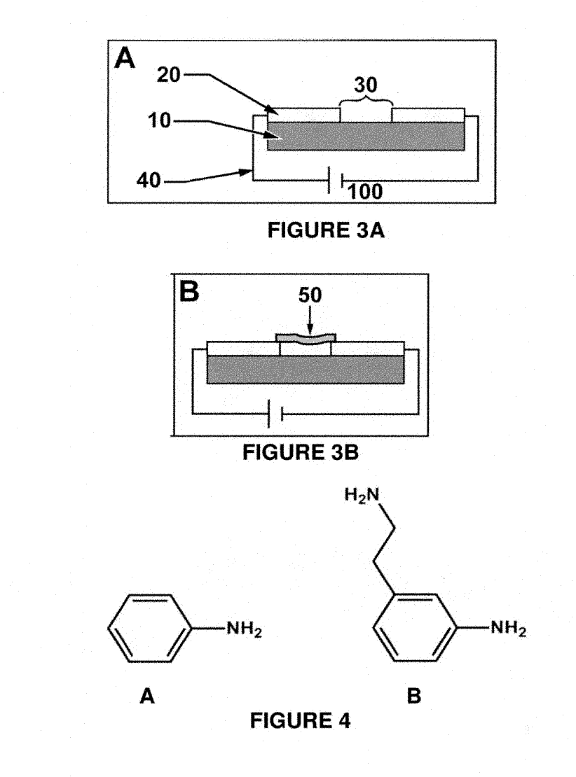

Referring now to FIG. 3, a schematic of a preferred embodiment of a PNMD is shown. This preferred embodiment has at least two electrodes 20 mounted on a cleaned silicon substrate 10. A preferred method of cleaning the substrate comprises placing the substrate in a 1% solution of an alkaline cleaning solution, preferably Micro-90® (International Products Corporation, Burlington N.J.) which is a concentrated, proprietary composition of chelating detergent that contains ionic and non-ionic ingredients, and applying ultrasonic energy for about 10 minutes. The cleaning solution is then replaced successively by solutions of DI water, isopropyl alcohol, acetone and DI water, with sonication of each solution, exposure for about 20 minutes to a Piranha Solution (7 parts H₂SO₄ to 3 parts H₂O₂) followed by copious rinsing with DI water and storing the cleaned silicone substrates in DI water.

Electrical leads are bonded to the electrodes and the connection is encapsulated by an epoxy material to protect it from the electrochemical process. The electrodes 20, preferably made of a noble metal such as gold or platinum, are placed on the cleaned silicon substrate in the electrolyte bath separated by a gap 30 of 0.5 μm (500 nm) to 4.0 μm (4000 nm), preferably about 2.0 μm (2000 nm) and are connected to a power source 100 by leads or wires 40. The assembled substrate and electrodes in the electrolyte bath is then purged and blanketed by a N₂ atmosphere. Electrical energy is then applied to the electrodes starting at about 0.7-0.8V. The process results in the electrodes 20 being electrically connected by a polymer nanowire or carbon nanotube 50 of diameter 30-150 nm and length 2-10 μm which are electrochemically formed, depending on the bath composition.

The simplest configuration of the nanowire sensor is a resistive junction composed of two solid state electrodes between which conducting polymer materials are grown. FIG. 10 shows a schematic test setup for measuring I-V curves of a nanowire sensor 900 which includes a gas supply 902, a water supply 903, gas mixers 904, a 3-way valve 906 connected to a gas inlet 908 of a test chamber 910. The sensor device 912 to be tested is connected to a semiconductor parameter analyzer 914 and placed in the test chamber 910. The electron transport properties of the sensor change upon exposure to analytes such as CO₂, MMH, NO₂ or bio-molecules. The equilibrium driven analyte binding interactions (van der Waals and/or dipole-dipole in nature) with nanowires change the electronic density and current flow of nanowires. The current-voltage curves of a nanowire sensor are measured before and after exposing the sensor to a target analyte (CO₂, MMH, NO₂ or biomolecues). The change in conducting current (before and after) of the nanowire sensor is directly proportional to the concentration of the exposed analyte. Therefore, by measuring the change in conducting current before and after the sensor is exposed to an analyte, the concentration of the target analyte can be determined. This calibration can be stored locally in the sensor assembly or in some other storage medium for later look-up.

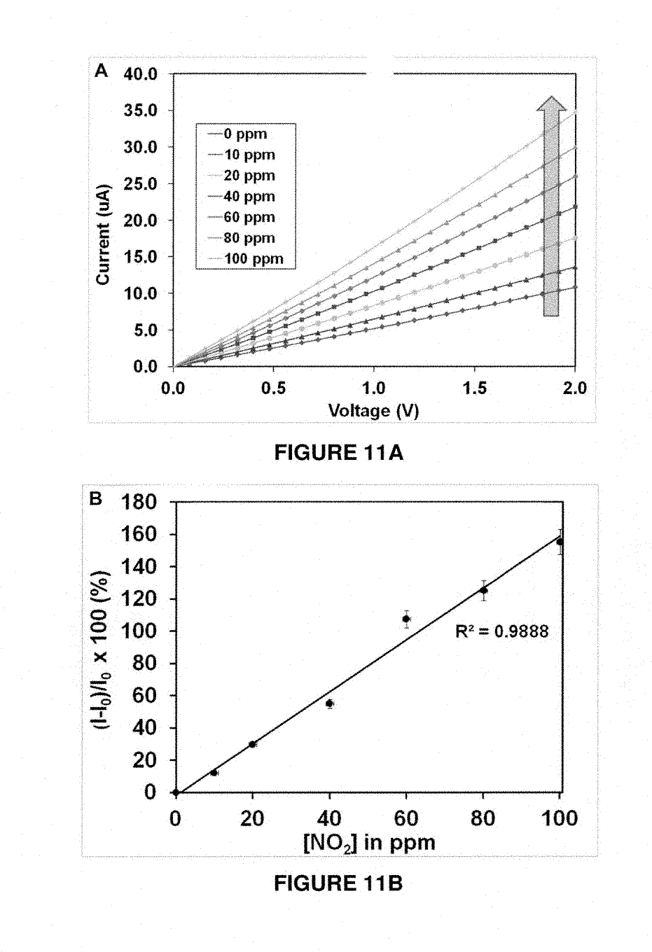

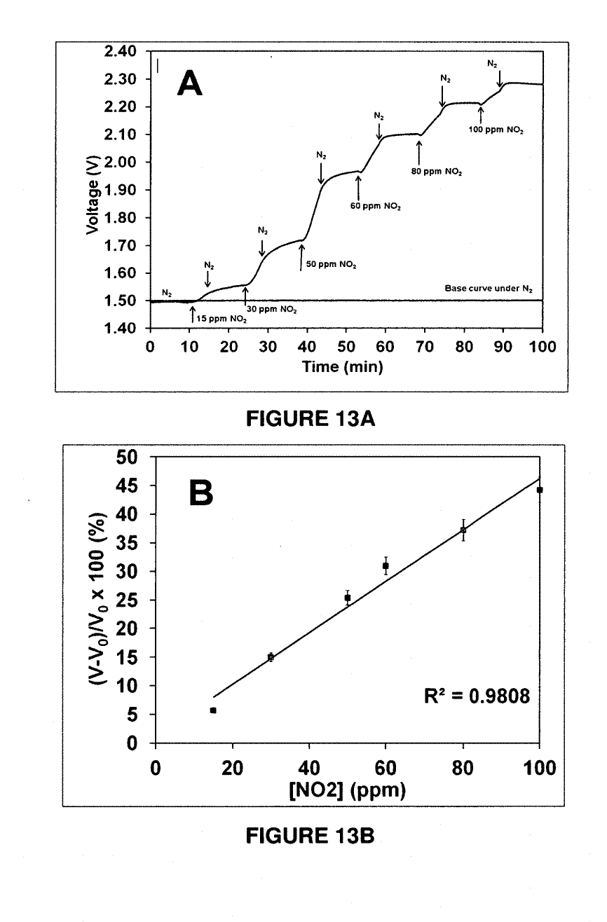

PNMD sensors in the presence of MMH and NO₂ at temperatures of −46° C., 0° C., 23° C., 40° C. and 71° C. in dry nitrogen (N₂) were tested. First, the PNMDs were tested for detecting NO₂ gas. The sensor signal responses were measured as current-voltage (I-V) curves and voltage-time (V-t) plots with an Agilent semiconductor parameter analyzer and a breadboard device (FIGS. 11 and 13). The I-V curves plotted with the Agilent semiconductor parameter analyzer showed that the PNMDs responded significantly when exposed to NO₂. The device current increased in positive direction with increasing NO₂ concentrations (0-100 ppm) and followed a linear trend line. Similar sensor response behavior was observed at all measured temperatures.

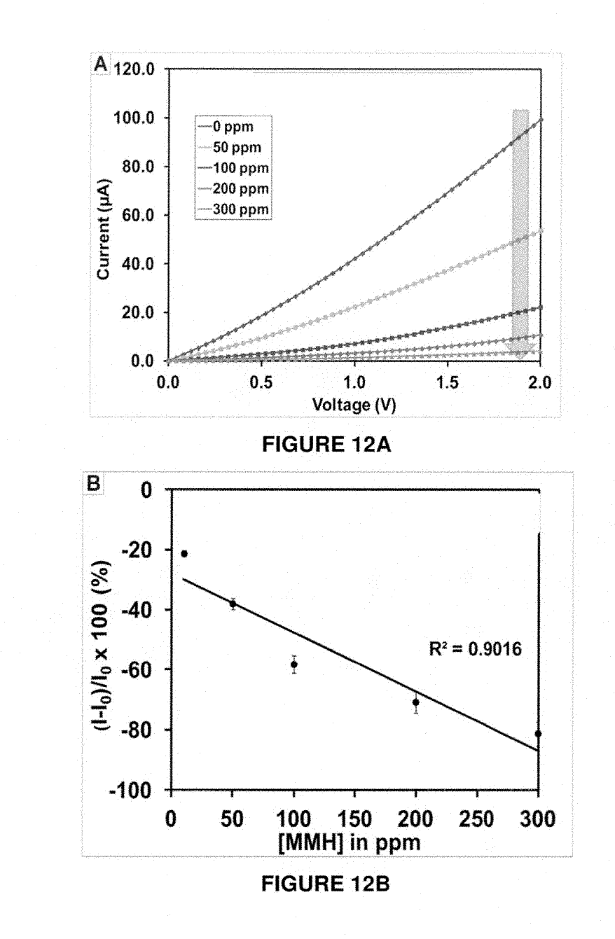

FIG. 12A shows I-V curves for detecting MMH at 23° C. measured with an Agilent semiconductor parameter analyzer. The PNMDs show significant response when exposed to MMH, and the device current decreased linearly as the concentration (0-300 ppm) of MMH increased (FIG. 12A) and followed a linear trend line (FIG. 12B). Similar sensor response was observed at other measured temperatures mentioned above.

FIG. 13 shows a representative voltage-time (V-t) plot measured by using a breadboard device at 40° C. for sensing NO₂. FIG. 13B shows that PNMD response increased to positive direction with increasing NO₂ gas concentration (0-100 ppm) due to oxidizing nature of NO₂. Similar NO₂ sensing responses were observed for the PNMDs at all measured temperatures.

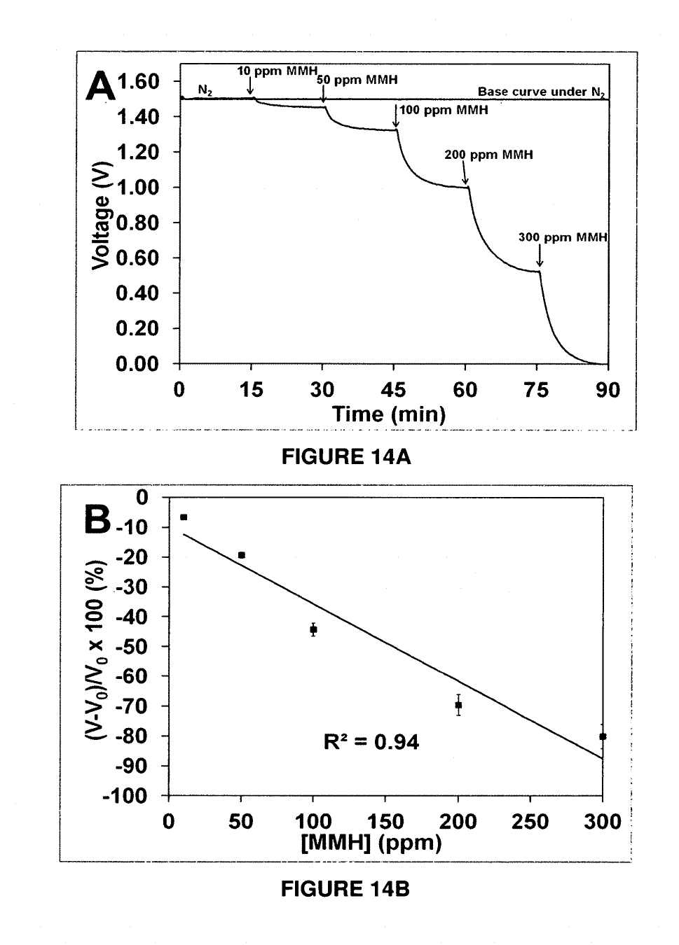

For sensing MMH (0-300 ppm), the sensor response (V) increased to negative direction with increasing concentration of MMH because of the reducing nature of MMH. FIG. 14 shows a representative V-t graph and a linear trend line plot at 40° C. for sensing MMH. Similar MMH sensing responses were observed for the PNMDs at all measured temperatures.

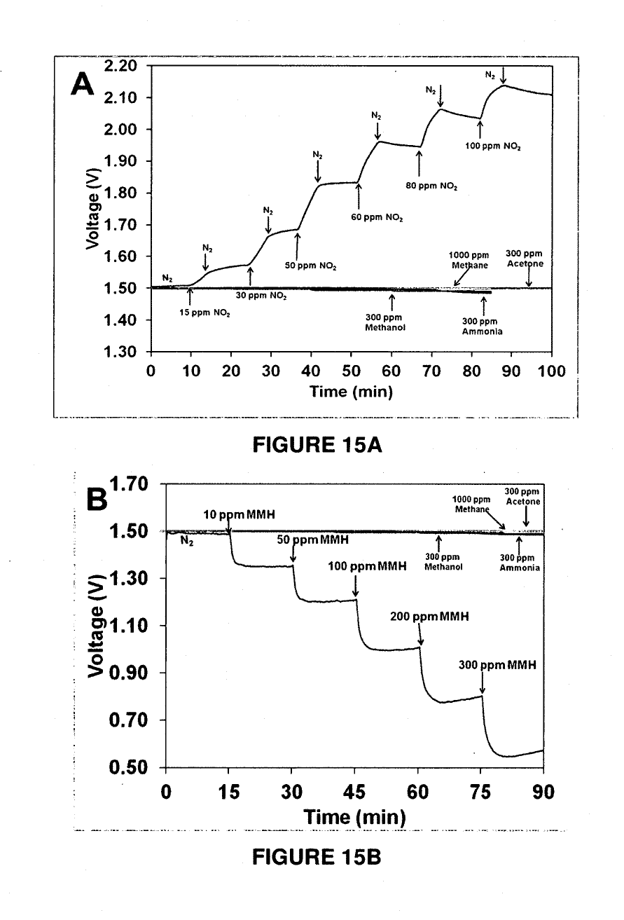

PNMD sensors can indicate trace leaks (≤50 ppm) of both MMH and NO₂ within minutes (<5 minutes) with high reliability, minimal cross-sensitivity, and minimal response to trace interference gases (FIG. 15). PNMDs show very promising long term operational stability (measured over six months), shelf-life, and tolerance to shock, and vibration.

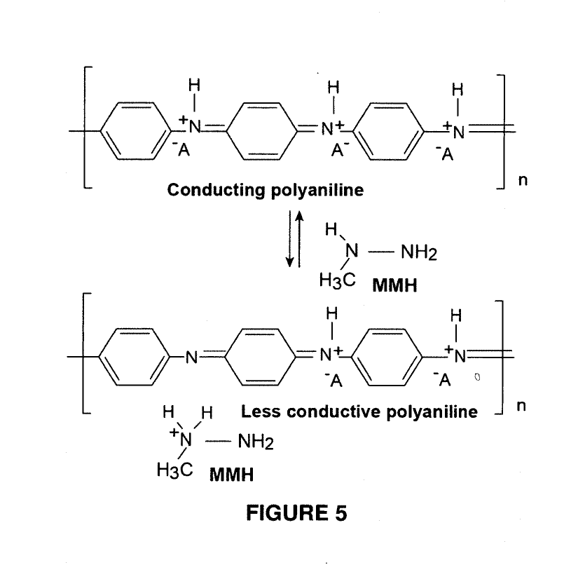

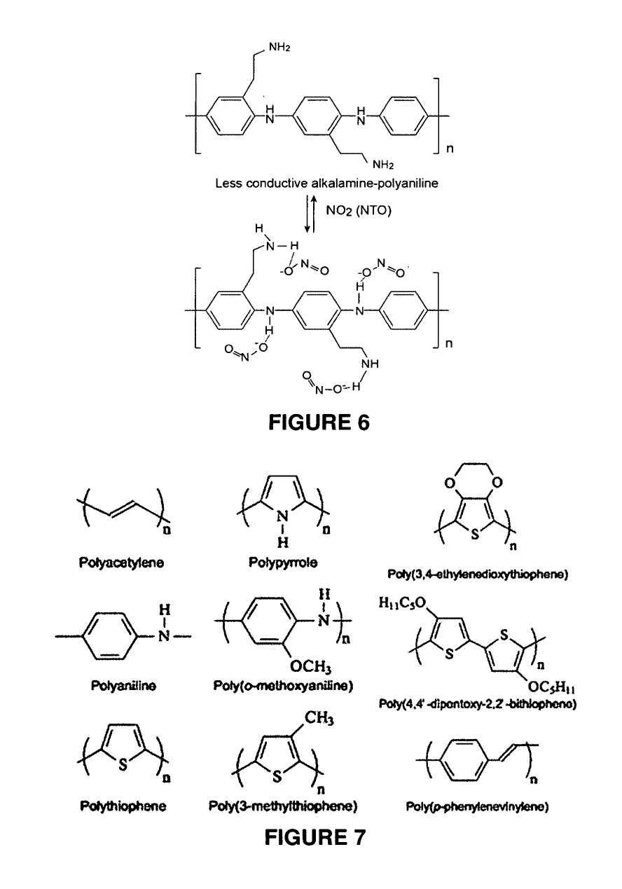

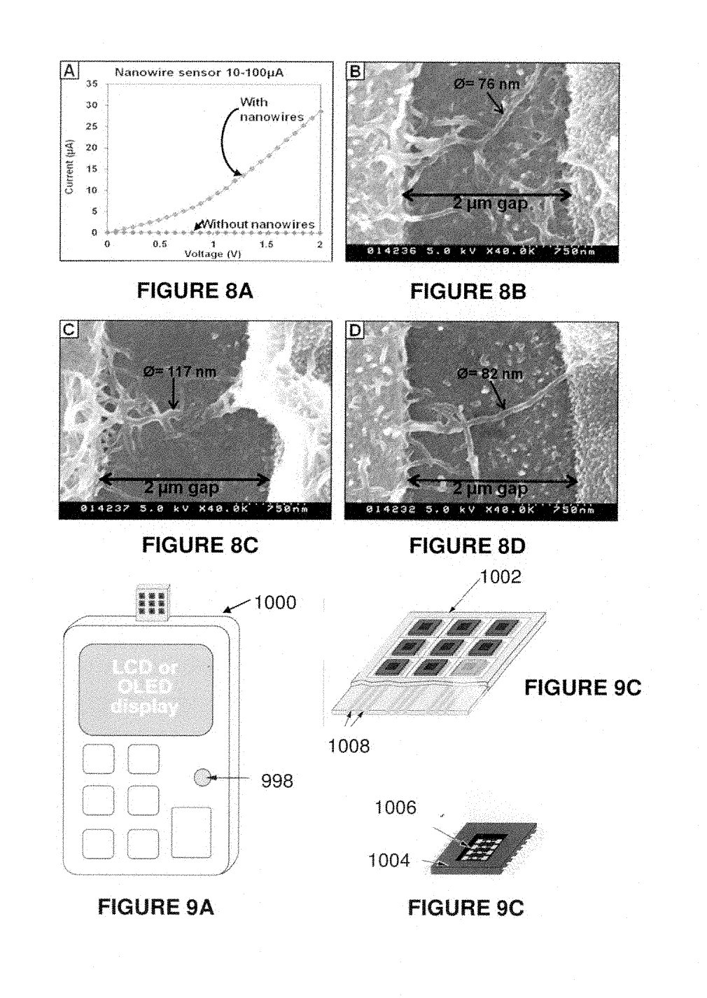

Chemical structure of polyaniline and its interaction with MMH are shown in FIG. 5. In this figure, the polyaniline is used to detect MMH. FIG. 6 shows the chemical structure of functionalized polyaniline and its interaction with NO₂. The novel process for the growth of polymer nanowires and the fabrication of both MMH and NO₂ sensors described herein was used.

### Carbon Dioxide (CO₂) Monitoring.

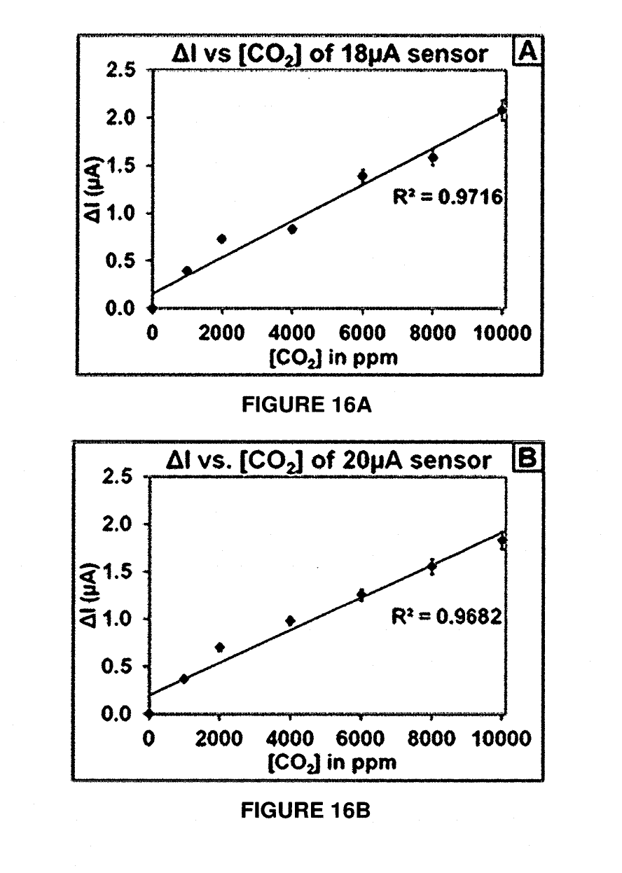

Through customization of polymer nanowires or carbon nanotubes by chemical synthesis, a nanowire or carbon nanotube sensor for detecting the environmental and subsurface CO₂ has been developed. The customized nanowire or carbon nanotube sensors detect CO₂ reversibly in the 0 ppm to 10,000 ppm range (FIG. 16) with response time of 2 minutes and reversing within 30 minutes in the temperature range of 10° C. to 60° C. and over 0% to 80% relative humidity. Using a DC intensity measurement system, CO₂ concentrations as low as 25 ppm were detected.

Polymer Nanowires Formation.

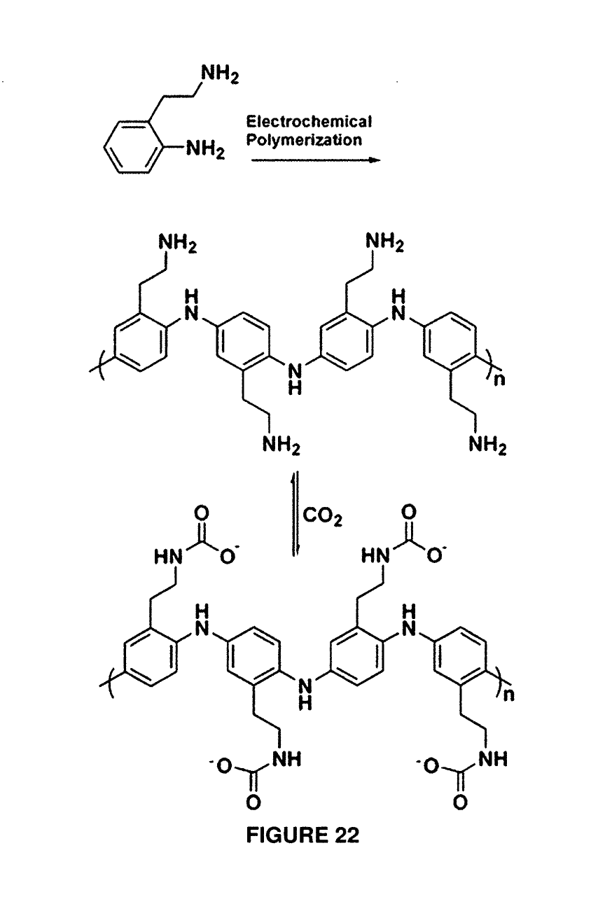

Alkyl amine-modified polymer nanowires for selective and sensitive CO₂ detection were prepared. First, alkylamine functionalized aniline monomer was synthesized by chemical reactions and this monomer was used to create alkyl amine-modified polymer nanowires using template-free electrochemical method. The chemical structure and its interaction with CO₂ are shown in FIG. 22. The formation of the carbonate upon CO₂ interactions with the amine groups of the nanowire imparts this selectivity. To the best of the applicants' knowledge, the amine-modified aniline monomer is a unique compound. The formation of the carbonate is a reversible reaction. Thus, the sensor is reversible and can detect both increases and decreases in CO₂ levels.

In addition, the novel growth process of creating polymer nanowires disclosed herein is unique. Six different electrolyte systems (formic acid, acetic acid, perchloric acid, hydrochloric acid, phosphoric acid and nitric acid) have been investigated with varying concentrations (0.2-1.0 M) in deionized water for the growth of polymer nanowires using three-step electrochemical method. These electrolytes offer specific counter ions, ionic strength, polarity, and acid strength (pKa) that play a critical role during nanowire growth.

Initial attempts to grow polymer nanowires were conducted using formic acid (HCOOH) at room temperature. Solutions of 0.2 M aniline (monomer) in 0.8 and 1.0 M HCOOH were prepared. Using the 1.0 M HCOOH solution, after the electro-polymerization process was completed, the Si chip devices were examined under a microscope with 45× magnification. The visual examination showed no growth and the device appeared to be unchanged from what it was before the electrochemical process. This was confirmed by current-voltage (I-V) characteristic measurements. The same results were obtained for the 0.8 M solution of HCOOH so lower concentrations were not attempted. The same results were observed when using acetic acid (CH₃COOH). The conclusion was that organic acids in general are poor electrolytes for the electrochemical growth of polymer nanowires.

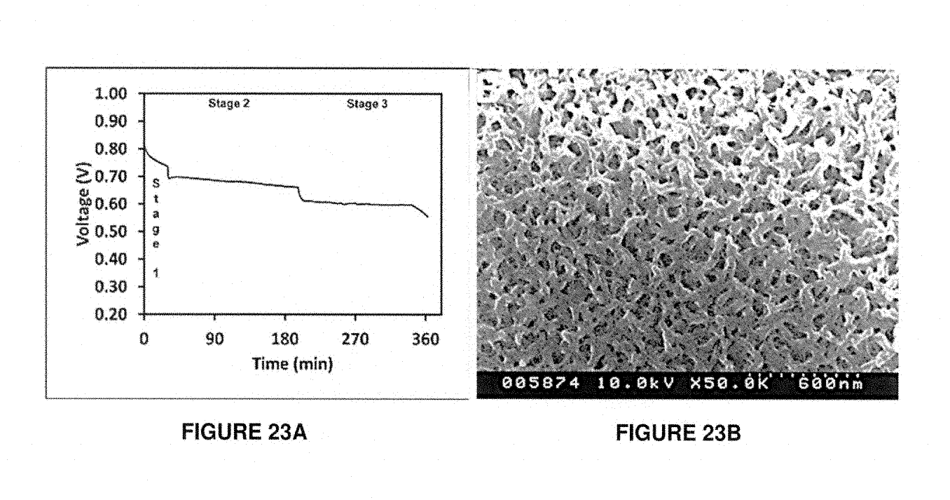

All five concentrations (0.2 M, 0.4 M, 0.6 M, 0.8 M and 1.0 M) of perchloric acid (HClo₄) showed varying degrees of growth. All four concentrations of HCl and HNO₃ electrolyte solutions (0.2 M, 0.4 M, 0.6 M and 0.8 M) showed varying degrees of growth. All of the inorganic acids resulted in successfully grown polymer nanowires (˜50-150 nm diameter and ≥2 μm length) in the concentration range (0.2-0.6 M) in various degrees. In particular, 0.2 M aniline monomer in 0.4 M HNO₃ electrolyte solution produced best polymer nanowires with a porous nano-network, spanning the gap and connecting the metal electrodes, as shown in FIG. 23.

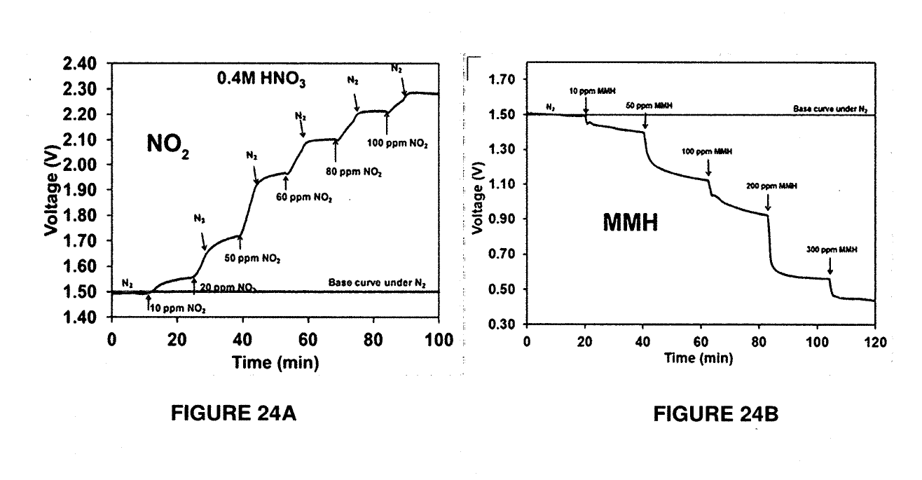

Polymer-nanowires sensor devices fabricated using the above mentioned inorganic acids including HNO₃ electrolyte solution were tested by exposing to analyte gases such as toxic nitrogen dioxide (NO₂) and monomethyl hydrazine (MMH) and their performance was evaluated. The Polymer-nanowires sensor devices grown with 0.2 M aniline monomer in 0.4 M HNO₃ electrolyte solution showed very sensitive and significant responses to six different concentrations of NO₂ (FIG. 24A) and also to MMH (FIG. 24B). Even at high concentrations, the device showed a clear difference in response compared to a closest concentration exposed.

Based on an evaluation of all the PNMDs from these different inorganic acid sources, all the acids that produced devices are responsive to analyte molecules/gases to some extent. But in terms of magnitude of response, sensitivity and stability during testing the 0.2 M aniline in 0.6 M HNO₃ electrolyte solution-based sensor devices appear to be the best with 0.2M to 0.4M HNO₃ electrolyte solution being preferred for the growth of these amine functionalized polyaniline nanowires.

Both the electrolyte system and its concentration were optimized to achieve high quality polymer nanowires with diameters ranging from 30 nm to 150 nm, length ≥2 μm with a highly porous nano-network morphology, resulting in high surface area, highly reactive sites and enhanced response and sensitivity for detecting CO₂.

The concentration effect of amine functionalized aniline monomer (0.1-1.0 M) was investigated in an electrolyte system (0.4-0.6 M nitric acid). The optimized monomer concentration was found to be 0.2-0.4 M in a nitric acid (0.4-0.6 M) electrolyte system to obtain the above mentioned high quality polymer nanowires.

The quality of polymer nanowires was further optimized by applying very low-level current (12-50 nanoampere) and slow growth mechanism over a period of time (4-6 hours). The high-quality polymer nanowires obtained in this process mentioned above were confirmed by scanning electron microscope (SEM) analysis, current-voltage (I-V) measurements and evaluating CO₂ sensor performance. This novel process was used for the fabrication of other sensors.

### Fabrication of Carbon Nanotubes (CNT) Biosensors—

A 3% carboxylic acid-functionalized single-wall CNT solution was prepared in several different concentrations—
(a) 0.1 mg/mL comprising 10 mL Dimethyl formamide (DMF)+1 mg CNT (aliquot 1 mL per vial for single use),
(b) 0.02 mg/mL comprising 5 mL DMF+1 mL of (a) above (0.1 mg/mL),
(c) 0.01 mg/mL comprising 10 mL DMF+1 mL of (a) above (0.1 mg/mL), and
(d) 0.005 mg/mL comprising: 10 mL DMF+0.5 mL of (a) above (0.05 mg/mL).
   Each sample was sonicated for ˜30 min prior to use to eliminate aggregates.

The electrode arrangement as described above was connected to a function generator and oscilloscope set to deliver 1.5 MHz at 2V for dielectrophoretic alignment of CNTs in the 2-micron gap between two metal electrodes mounted on the substrate and 20 μL of CNT solution was placed into the gap of the device.

The device was connected to the function generator and voltage was applied for 30-120 seconds (selected as necessary to deposit CNTs bridging the device gap) followed by rinsing with deionized (DI) water to remove excess DMF and the device was allowed to dry completely at RT. Alternatively, a clean absorbent wipe can be applied to the edge of the device to wick the DMF from each device and then the device was air dried at room temperature.

As a quick check if sufficient CNTs were deposited, the electrical resistance of the dried device was determined using a multimeter.

The devices with CNTs spanning the gap were then annealed in a closed oven at 200-250° C. for 1 hour followed by cooling for about 30 minutes to reach ambient temp.

I-V curves across the device were then generated. An increase in current when compared to the non-annealed device should have resulted as a result of CNTs contacting the gold electrodes now being annealed to the surface.

A 1-pyrenebutyric acid N-hydroxysuccinimide ester (PBASE) solution comprising of 1.5 mL of 6 mM PBASE (MW=385.41 g/mol) and 20 mL of DMF=46.25 mg 5 mM PBASE was prepared, covered by foil (because PBASE is light sensitive) and stored at −25° C.

The device was then placed in 2-3 mL of the PBASE solution for 30 min at RT in the dark, followed by washing with MilliQ™ H₂O (the H₂O was autoclaved and neutralized prior to use because PBASE has a tendency to bind and react with many contaminants) and incubation for 5-10 min (or until the devices are completely dry) at the very minimum at 40° C. I-V measured across the device again will then displays a current decrease after PBASE binding.

Antibody and antigen samples in appropriate media were then prepared. 4 μL of a selected antibody solution was placed on top of the gold electrodes of each device and incubated at 37.5-40° C. for up to 60 minutes (or until completely dry if necessary). During the incubation, the device can be kept in a closed container (such as a Petri dish) with wet Kim Wipe™ or cotton pad to maintain humidity inside the container so the antibody solution will not become dry.

Passivation buffer solutions were prepared comprising:
1. 0.1% Tween 20=500 μL Tween 20+499.5 mL MiiliQ H₂O,
2. 6 mM 6-Mercapto-1-hexanol (MCH) (MW=134.24 g/mol; d=0.981 g/mL)=410 uL MCH+500 mL MilliQ H₂O, or 6 mM MCH=82.10 uL MCH+99.92 mL MilliQ H₂O, and
3. 0.1 mM Ethanolamine (EA)=1.5 mL EA+498.5 mL MilliQ H₂O.

The device was submerged in 0.1 mM EA for 30 min at RT, then in 0.1% Tween 20 for 30 min at RT followed by submerging in 6 mM MCH for 1 hr at RT. The passivated device was then rinsed with MilliQ H₂O and dried using a Kim Wipe™ and/or air dried at RT for 1 hr.

If not used immediately the passivated devices should be wrapped in parafilm and foil and stored at −25° C.

### Measuring the I-V Characteristics

Before using the CNT Biosensor for analyte detection, the I-V properties of the passivated device should be determined to provide a base curve. To use the sensor a diluted mixture of antigen/media is applied to the sensor and the sensor is incubated for at least 20 minutes at 37.5-40° C., followed by determining the I-V characteristics of the treated sensor. This can be repeated using different concentrations of the antigen/media.

### Detection of Disease Biomarkers.

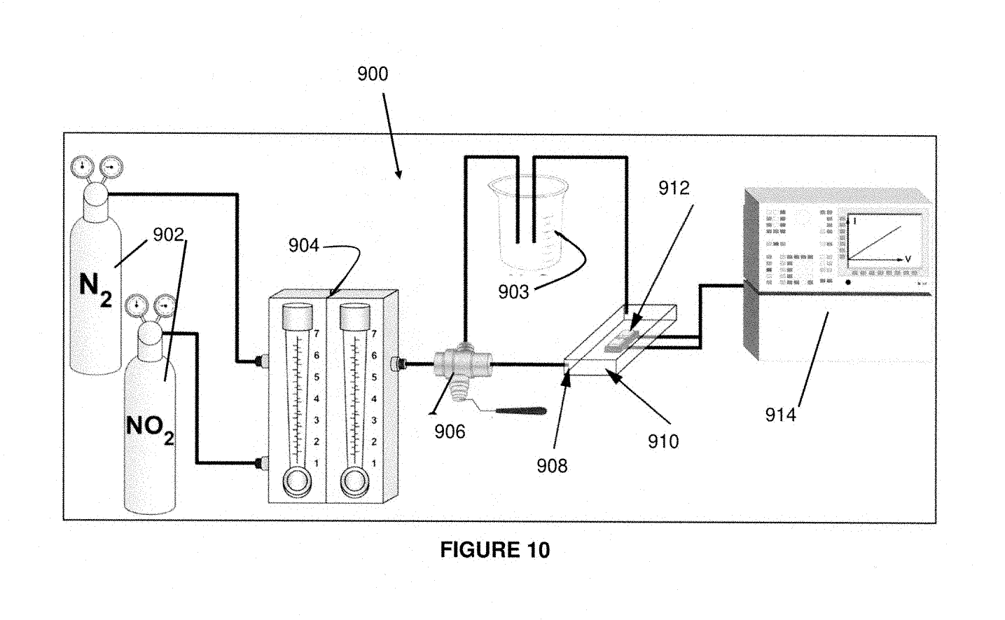

The sensors described herein can also be used for detection of disease biomarkers. Referring to FIG. 9, a general schematic for a biosensor for use in diagnostics is shown. A nanowire biosensor array is mounted on a biosensor chip. One or more chips are mounted on a card with electrical contacts. Each of the chips can be designated for detection of a different disease biomarker (for an expanded survey of tests for, e.g. Alzheimer's (see below), Parkinson's, Hepatitis, Cardiac disease, etc.) or each of the chips can be designated for the same disease biomarker (for additional accuracy). The card is then inserted into the hand-held device shown in FIG. 9A for analysis of the data collected from the biosensor chips and card.

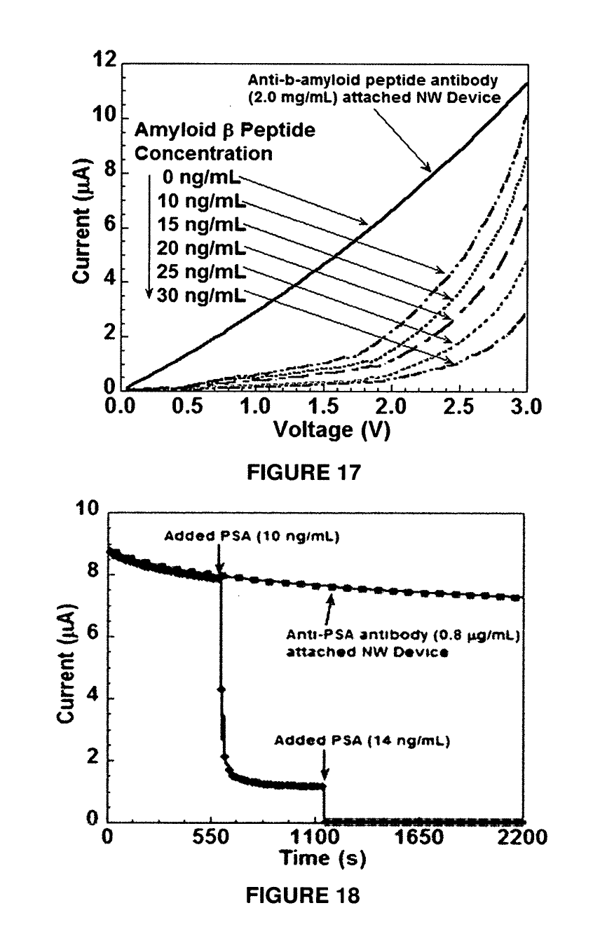

Another preferred embodiment is a porous polymer nanowire or carbon nanotube platform-based sensor for early diagnosis of Alzheimer's disease (AD) by detecting AD-associated biomarkers. Conducting polymers or carbon nanotubes modified with covalently attached antibodies specific to different AD biomarkers such as different forms of Aβ (monomers and oligomers) as capture and transducing agents for an electrochemical-based biosensor were used in the sensor. Nanowire or nanotube devices detect 36 pM for the Aβ oligomer and sub-pM for the Aβ monomer. This is approximately three orders of magnitude better than what can be achieved using the same antibodies in enzyme-linked immunosorbent assay (ELISA) or blot tests for Aβ detection (1-10 nM). Antibodies are attached to nanowires or nanotubes via amide coupling using N-hydroxysuccinimide. Standard current-voltage (I-V) curves were obtained when the anti-Aβ42 sensors were tested with a semiconductor parameter analyzer. FIG. 17 shows the I-V curves of an anti-Aβ42 peptide antibody-attached sensor exposed to varying concentrations of Aβ42 in phosphate buffered saline (PBS) solution for 5 min. at each concentration. A significant change in the I-V curve of the anti-Aβ42 peptide antibody-attached sensor was observed after successive exposures of Aβ42. All sensors responded to the introduction of Aβ, as expected. This antibody-based nanowire or nanotube sensor exhibited much higher sensitivity than the ELISA and Western-blot tests.

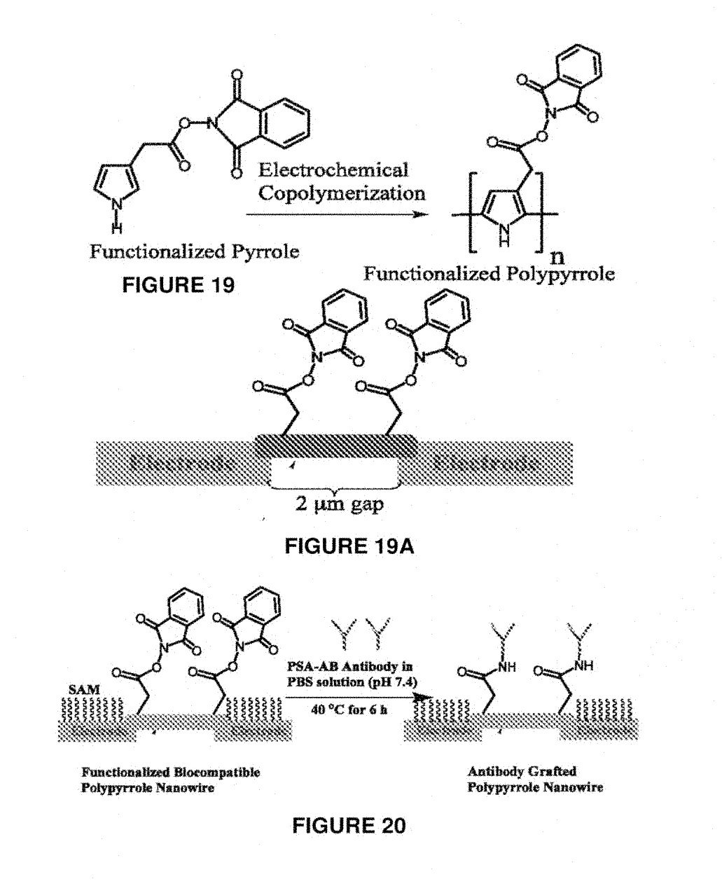

Referring now to FIG. 19, for the development of biosensors to detect Alzheimer's disease (AD), N-hydroxyphathalo-succinimide or N-hydroxysuccinimide functionalized pyrrole monomer was synthesized and created N-hydroxyphathalo-succinimide or N-hydroxysuccinimide functionalized polypyrrole nanowires by electrochemical method. FIG. 20 illustrates and antibody conjugation followed the above synthesis.

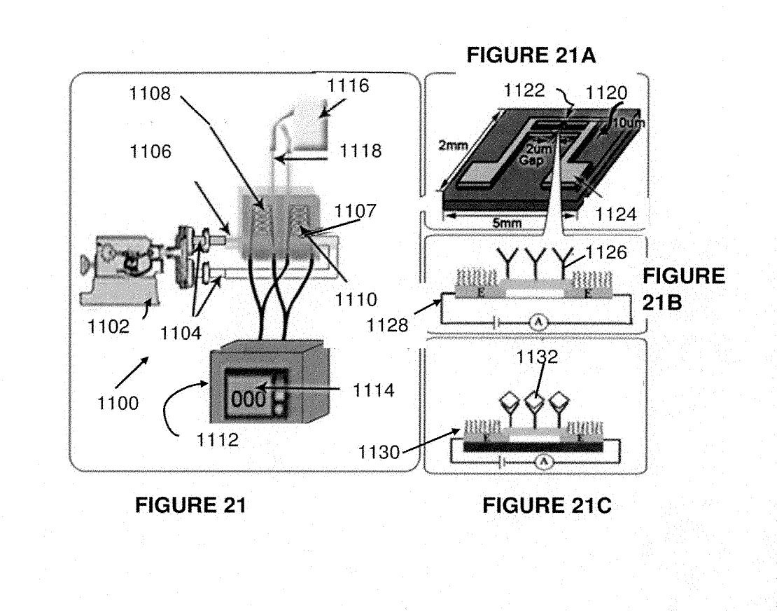

FIG. 21 is a schematic diagram showing a testing device with enlarged views illustrating a polymer nanowire assembly with an PSA-AB antibody attached and the interaction of the antibody with corresponding disease biomarker antigen or protein for detecting disease. The testing device 1100 includes a pump 1102 connected delivery syringes 1104 which feeds a test sample through a sample inlet 1106 into a test unit 1107 containing a sample device (PNMD) 1108 and a reference device (PNMD) 1110. The sample device 1108 and the reference device 1110 are connected to a signal processing control unit 1112 which has a display 1114 for showing results of the test. Waste sample material exiting the test device 1107 is passed through a scrubber 1116 to a waste outlet 1118. Also shown is an enlarged view of a chip device 1120 which includes a conducting polymer nanowire 1122 between left and right connecting pads 1124. The same construction chip device 1120 is used for both the sample device (PNMD) 1108 and the reference device (PNMD) 1110 with the difference being that the sample device (PNMD) 1108 is labeled with PSA-AB antibody 1126 as schematically shown in first and second further enlarged views 1128, 1130, the second further enlarged view 1130 schematically illustrating attachment of a PSA antigen 1132 after incubation at room temperature for 2 hours.

A polymer nanowire or carbon nanotube sensor device for the detection of prostate cancer biomarker PSA (prostate specific antigen) is another preferred embodiment. The response time of nanowire- or nanotube-based sensors was evaluated by detecting current changes as a function of time. FIG. 18 shows the current vs. time response for a nanowire or nanotube sensor functionalized with anti-PSA antibodies that was exposed to a constant bias of 2.5 V. A much more significant change in current was observed when small concentrations of the antigen were added. In all cases, the response was observed and stabilized within a few minutes.

Using the procedures described above, sensors were characterized and tested and their performance, such as response, sensitivity, selectivity and reproducibility for the detection of toxic metals in phosphate saline buffer solution (PBS) and biofluids (urine, saliva) and detection of disease biomarkers (proteins) in PBS, artificial cerebral spinal fluid (aCSF) and clinical CSF media were evaluated

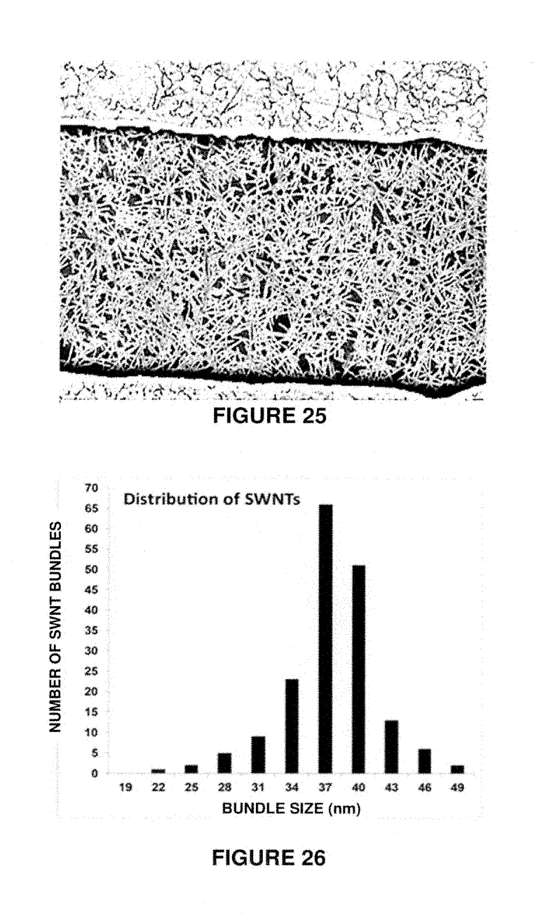

Single-wall CNTs with an average bundle size of 37 nm spanning the 2 μm gap of the microelectronic Si-chip sensor devices were produced. FIG. 25 is a scanning electron microscope (SEM) image of the CNTs within the 2 μm gap of a microelectrode in a sensor device produced according to the procedures described herein. FIG. 26 is a histogram of the bundle sizes of the CNTs shown in FIG. 25. The single-wall carbon nanotubes (SWNTs) allow any applied current to pass across a gap. The changes in the current passing across the SWNT are the basis of detecting target biomolecules, toxic heavy metal-ions or other toxic/cancerogenic chemicals. The protocols set forth herein provided superior sensor fabrication yield and reproducibility.

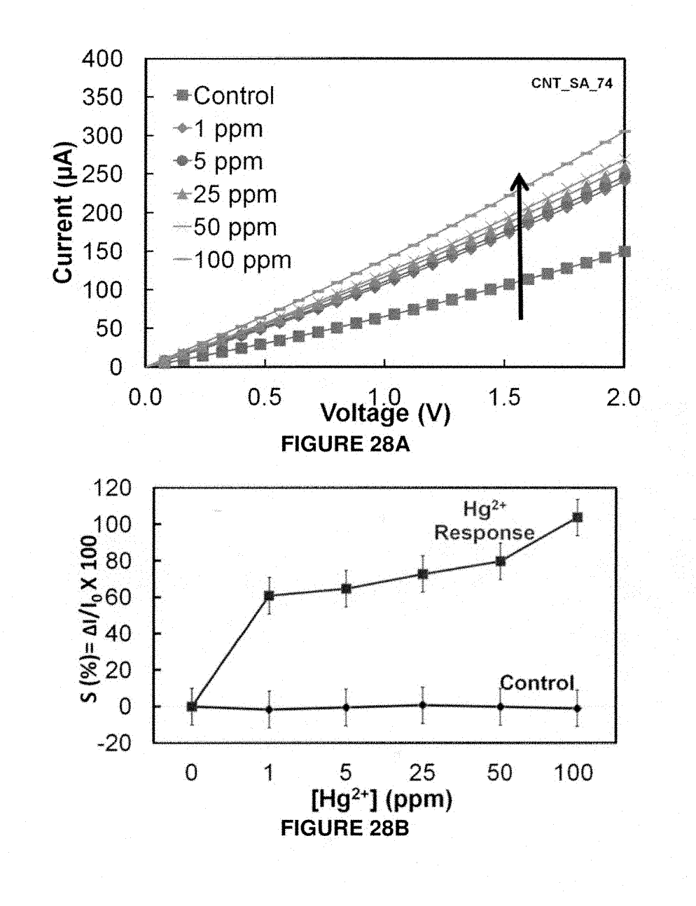

The performance of mercury sensors produced as described herein were tested and evaluated for detecting the presence of mercury (Hg²⁺) ions in water, urine and saliva. FIG. 28 shows the measured I-V curves of a representative mercury sensor device. The acquired I-V curves show an upward trend in current when the device was exposed to varying concentrations of Hg²⁺ ions. The derived calibration curve (percent response versus Hg²⁺ ion concentration; FIG. 28B) of the device also shows the upward trend in response with increasing concentration of Hg²⁺ ion. The sensor showed a significant response compared to its control counterpart. The observed response was approximately 70.0±3.5% at 1 ppm and 103.0±5.2% at 100 ppm. A t-test was conducted and the p-value with α=0.05 and obtained p=0.006 was calculated. Based on the p-value, it can be concluded that the results were statistically significant.

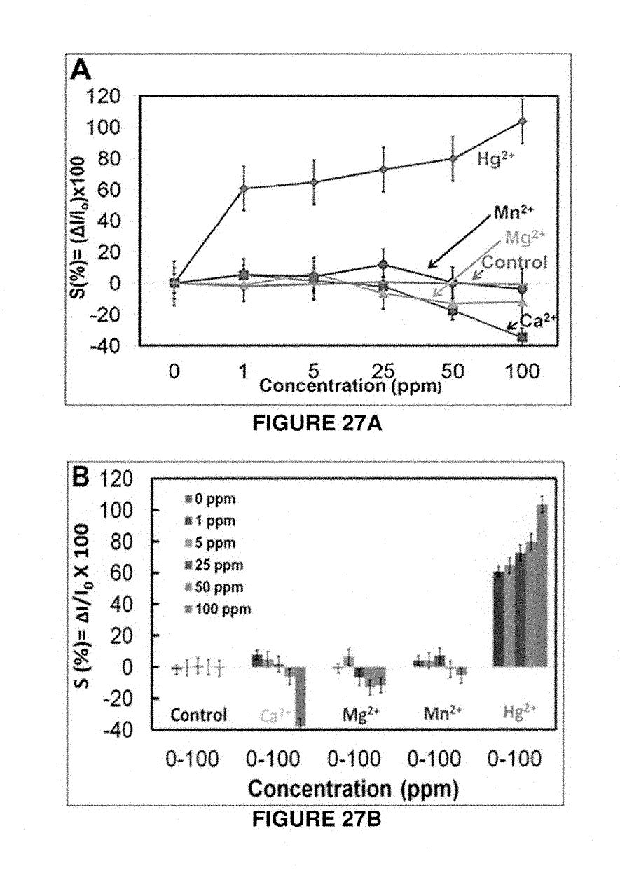

Mercury sensor responses were tested and evaluated with different interfering ions in water to establish its sensitivity and specificity toward Hg²⁺ ions. The sensor responses for calcium (Ca²⁺), manganese (Mn²⁺), magnesium (Mg²⁺), mercury (Hg²⁺) and a control in water are shown in FIGS. 27A and B. These results show that the device response for Hg²⁺ ions is significantly stronger than the response to the interfering ions. The overall response for Hg²⁺ ions is 100× stronger (an average p<0.01 by t-test) than the average maximum response for the interfering ions at 100 ppm. This comparison suggests that our mercury sensor is relatively specific and sensitive to Hg²⁺ ions compared to the other ions in water.

Multiplex array-based biosensor devices (proteins) were also fabricated and demonstrated the capability of detecting Alzheimer's Disease (AD) associated biomarkers in PBS, artificial cerebral spinal fluid (aCSF) and cerebral spinal fluid (CSF) samples. After nanowire growth and surface passivation, the devices were conjugated to commercial AD-specific antibodies (Abs) such as amyloid beta 1-42 (Aβ1-42), tau and p-tau Abs. The biosensor response to each AD biomarker was evaluated using 4 or 5 devices.

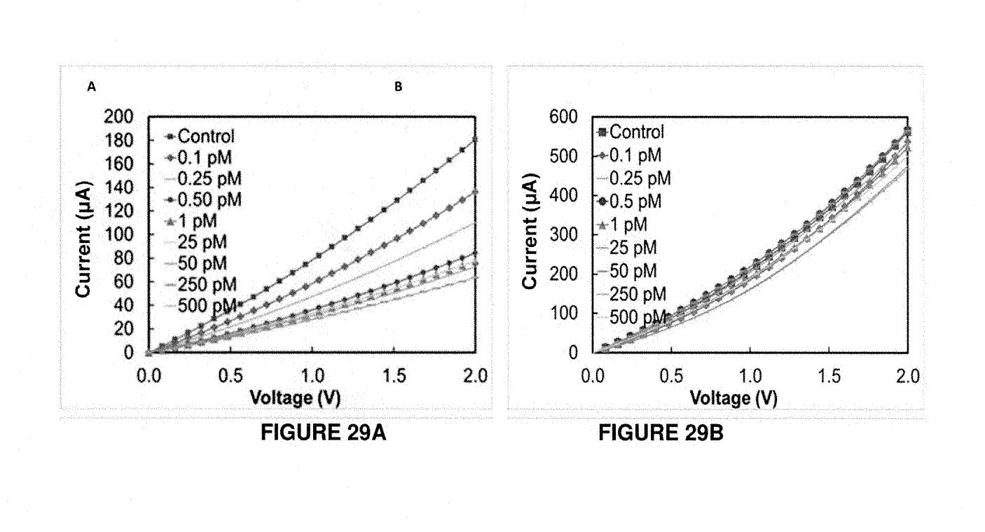

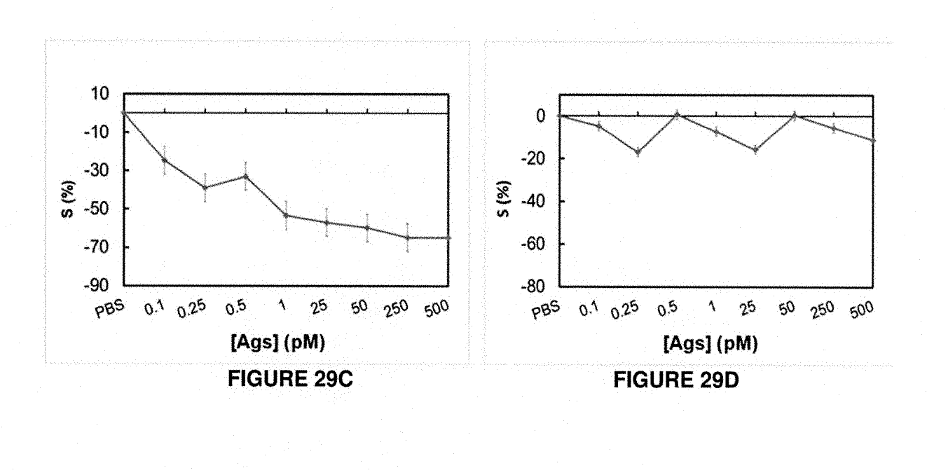

FIG. 29A-FIG. 29D show the response of biosensor devices conjugated to Aβ1-42 Abs and exposed to AD biomarker antigen (Ag) Aβ1-42 in PBS. The I-V curves show the current-voltage profile of Nanowire Sensor Array-based Assay for Early Diagnosis of Alzheimer's Disease (Adnos) devices conjugated with Aβ1-42 Ab and exposed to varying concentrations of Aβ1-42 Ag in 1×PBS (pH 7.4 at room temperature) in increasing order of concentration starting from 100 femtomolar (fM) to 500 picomolar (pM). A clear and significant decrease in current compared to the first exposure with no Ag (control) was noted. The sensors consistently responded to each concentration studied with a successive decrease in current as the concentration of the Aβ1-42 antigen (Ag) increased. This indicated that the devices were consistently sensitive to AD-specific biomarkers in the femtomolar to the picomolar range. The biosensor device responded by showing a downward trend in current response of −21±1.05% at 100 fM Ag which decreased sequentially to −63±3.15% at 500 pM Ag in PBS. The I-V curve (FIG. 29B) shows a control device with no antibodies (Abs) conjugated to polymer nanowires. There is almost no clear response trend when exposed to the serially diluted Ag in PBS. The biosensor devices showed a statistically significant response of 0.002 p value as depicted by t-test. The concentration range of 50 to 110 pM is significant because the clinically relevant concentrations of the widely accepted Aβ biomarkers are encompassed within this range.

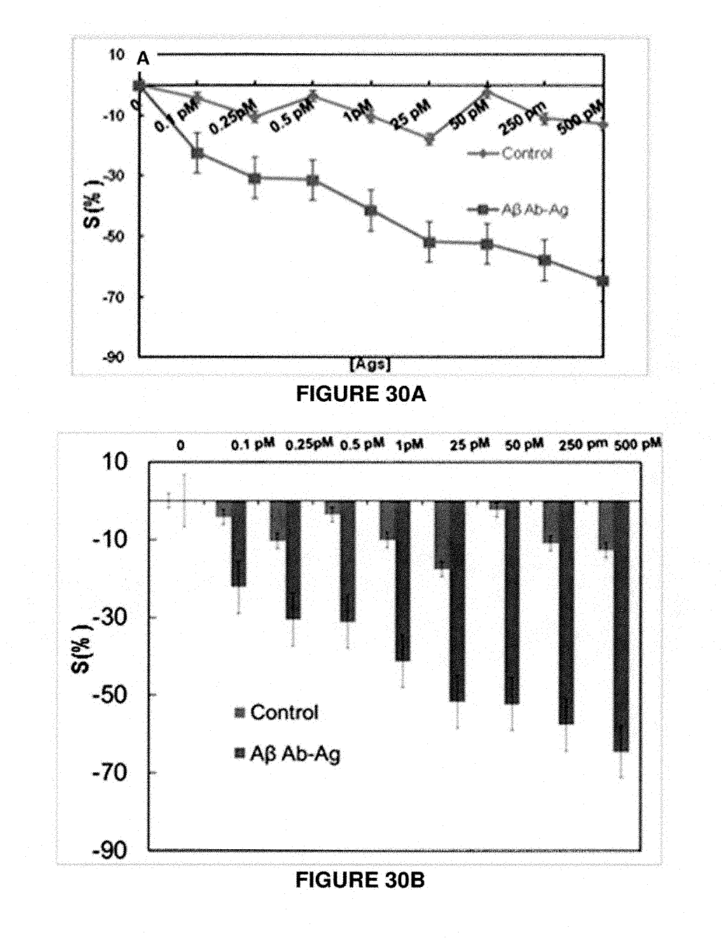

FIG. 30A and FIG. 30B show the calibration curve derived from the I-V curve showing the response of the biosensor devices compared to the controls. The lower line in FIG. 30A and the longer bars in FIG. 30B shows the average response of multiple biosensor devices conjugated with Aβ1-42 Ab. The other curved line and bars show the response of control devices where the nanowires have no Abs conjugated to them. The response at each concentration of Ag is significantly different from the controls. At 100 fM of Aβ1-42 peptide antigen, the average response of the biosensor devices was −22% and at 500 pM the response decreased to −66%. The control devices showed a response of −4% at 100 fM of Ag with no clear decrease in current response. At 500 pM, it was −12%. The response between control and response was statistically significant with a p value <0.001 as determined by t-test.

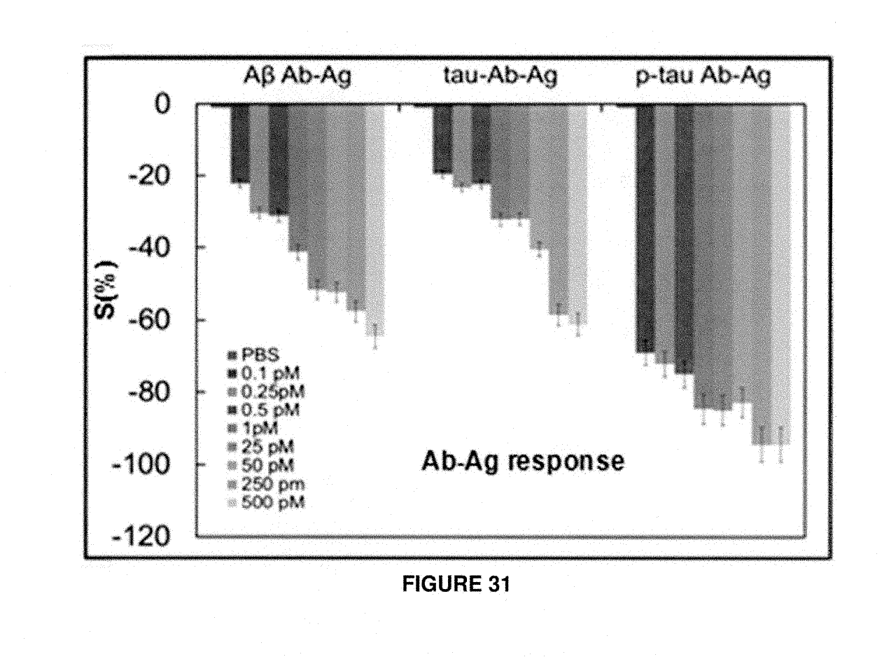

The response of tau and p-tau Abs conjugated biosensor devices was also tested and evaluated. The devices were exposed to a similar serial dilution of tau and p-tau Ag. The same downward trend in current compared to controls was observed. FIG. 31 summarizes the response of biosensor devices to each pair of Ab-Ag interaction. Average response from each pair of Ab-Ag interaction was calculated using 6 to 7 devices. Devices conjugated with Aβ1-42, tau or p-tau Abs were exposed to their corresponding Ag. The downward trend in current was seen with increases in corresponding Ag concentrations. In the case of Aβ1-42 Ab-Ag response, the response was almost linear from 100 fM to 500 pM. In the case of tau Ab-Ag response the downward current response was seen, but the response did not show any linearity between 100 fM and 500 fM. The current response further decreased at 500 pM. The highest response was seen in case of p-tau Ab-Ag in which, at 100 fM of p-tau peptide antigen, the response of the Adnos devices were −69% and at 500 pM the response decreased to −94%. In each case of Aβ1-42 Ag response, the difference was statistically significant as determined by t-test where p value=<0.005. Additionally, the sensor signals increased distinctly in the concentration regime from 50 to 110 pM. This concentration range is critical because the clinically relevant concentrations of the known Aβ biomarkers are encompassed within this range. In the particular case of p-tau Ab-Ag, the response at 100 fM was a sharp negative trend and the response appeared to plateau from 25 pM to 500 pM. This could be because the Abs was already saturated due to the Ab-Ag interaction, an aspect that will be optimized.

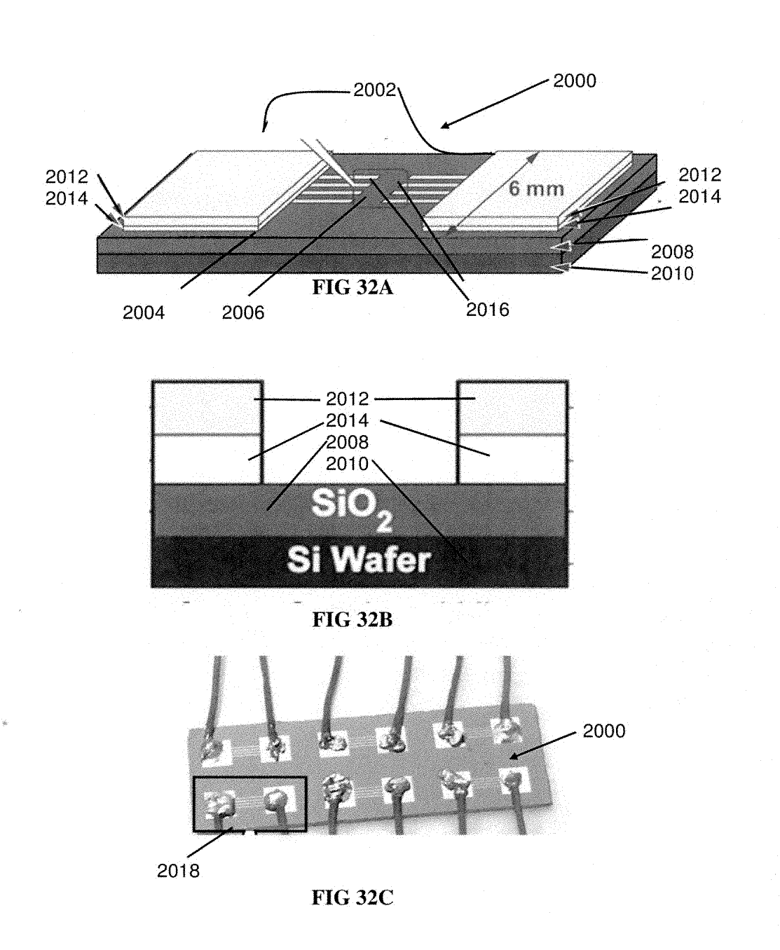

A further embodiment of a microelectrode device (MED) 2000 incorporating features of the inventions is shown in FIGS. 32A and 32B with 32A showing a top perspective view and 32B showing a side view of the microelectronic device. The MED comprises two conductive electrodes 2002 on a non-conductive substrate 2004 with a channel providing a gap 2006 between the electrodes 2002. The device shown is fabricated on a substrate 2004 preferably comprising Si/SiO₂ substrate wafer with a preferred 400-500 um SiO₂ insulating layer 2008 on a silicon wafer 2010 such as shown in FIG. 32B. Alternatively, the substrate can be selected from a broad range of inert substrates such as glass, SiO₂ glass, a plastic film/strip (such as Kapton), a non-conductive fabric, or paper. The conductive electrode material is preferably gold, copper, carbon, Ni, etc. In a preferred embodiment best shown in FIG. 32B each electrode comprise two conductive materials with an upper portion 2012 comprising gold and a lower portion 2014 comprising titanium. The electrodes 2002 can be fabricated via photolithography or other printing techniques including, but not limited to, screen printing, ink jet printing, or aerosol-jet printing. Each electrode 2002 is composed of a large square pad and preferably includes several (four or more) finger-like structures 2016 (not shown in FIG. 32B) extending toward the other electrode such as shown in FIG. 32A. The width of the fingers 2016 are typically 10˜200 um and the gaps between the fingers of two adjacent electrodes are typically 1˜20 urn. Alternatively, a third gate electrode (not shown) can be fabricated at the bottom of the wafer to form a field effect transistor structure. Prior to depositing the SWNT the device is thoroughly washed with hexane, acetone, IPA, DI and water using sonication in a solvent bath for 10 min each. The device 2000 is then further washed and activated with piranha solution, or alternatively cleaned using an ozone/reactive ion cleaner. FIG. 32C shows an example of an MED that has six (6) different pair of electrodes 2018, described further herein below for detecting multiple different biomaterials.

### Active Channel Fabrication.

Active materials, namely single-walled carbon nanotubes (SWNTs), are deposited in the gap areas between the fingers of two adjacent electrodes. Various commercially available brand/type of SWNTs can be used including, but not limited to, P2 SWNT or P3 SWNT (Carbon Solutions, Inc, Riverside Calif.)”, HiPco®SWCNT (Nanolntegris, Boisbriand, Quebec, Canada) or “CoMoCAT® CG65 SWNT” (SouthWest NanoTechnologies, Norman, Okla.) etc.). A preferred method for depositing the carbon nanotubes is using dielectrophoresis (DEP). To do so a SWNT solution is first prepared by adding SWNT, or derivatives of SWNT prepared by chemical functionalization, into a suitable solvent such as Dimethylformamide (DMF) or ethanol to form a suspension of 1 mg/mL which is then exposed to ultrasound (sonicated) in a sonicator bath for at least about 1 hr. The SWNT suspension is then centrifuged at 16000 rpm for 1 hr to remove any impurities and the top 90% of the supernatant is collected to provide a stock solution. The stock solution is then further diluted 10 ˜1000 times with the same solvent to provide the solution for DEP deposition.

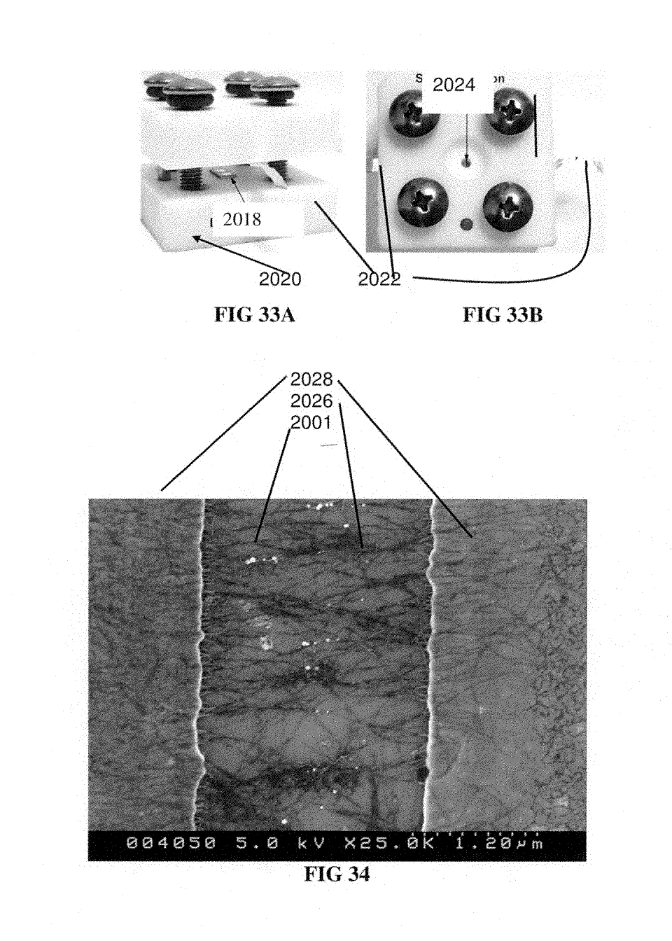

The MED described above is then placed in a Teflon® DEP deposition cell 2020 (FIGS. 33A and 33B) with electrically conductive leads 2022, such as aluminum or copper tapes or gold coated spring-loaded pins, connected to each electrode. The center part of the devices, mainly the finger structures is exposed to a round opening 2024 of around 2 mm. 20 uL of the above mentioned deposition solution is carefully added through the opening to avoid any air bubble building up between the device and the solution. The two electrodes are then connected to a wave-generator (not shown) and an AC electric wave is applied to the electrodes 2018, the electrical wave preferably having an amplitude of 2V˜15 V, and a frequency of preferably about 1.5 MHz delivered for 30 sec. to 10 min. After the DEP process, the device is washed with DMF, IPA and DI water and then annealed in an oven at about 200˜230° C. for 1 hr. An electron microscopy image of a typical device with SWNT 2001 bridging the 2 um gap 2006 between two finger electrodes 2028 is shown in FIG. 34. Alternatively, the SWNT can be deposited onto the device via (a) chemical absorption or (b) printing methods.

### Sensor Device Functionalization

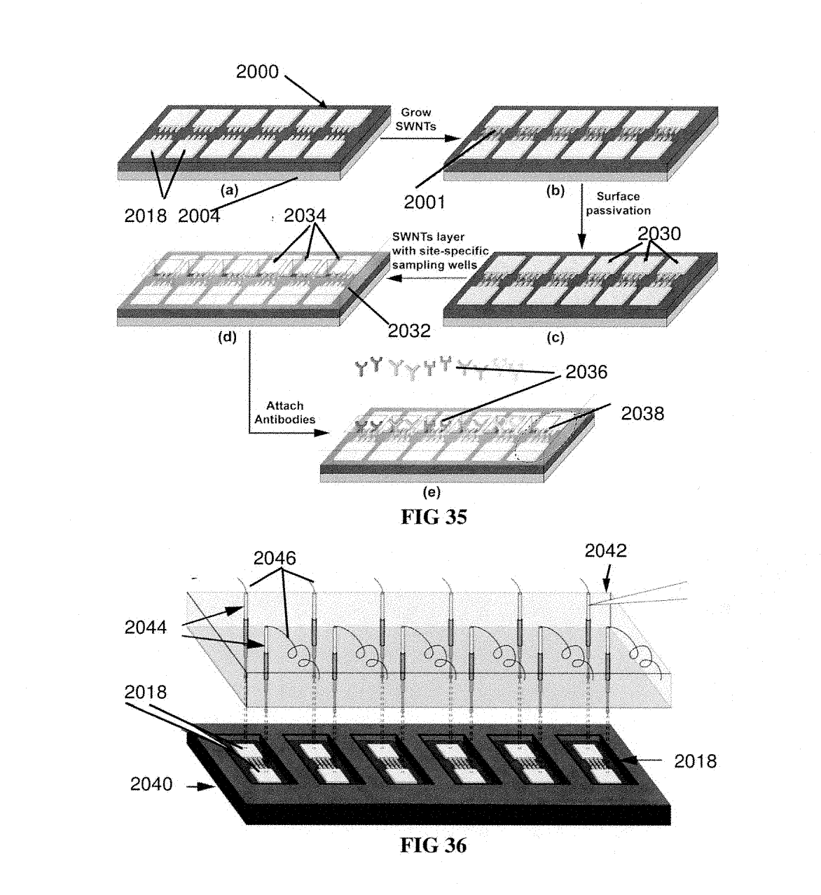

FIG. 35 illustrates the steps to activate SWNTs following deposition of the SWNTs 2001, in the gap 2006 between electrodes where (a) is the starting chip 2000 structure showing multiple pairs of microelectrodes 2018 (12 pairs), such as shown in FIG. 32A, and (b) schematically illustrates the chip 2000 after growing the SWNTs 2001. The microelectrodes 2018 are mounted on a substrate 2004. As an example, to functionalize the SWNTs 2001 with biomarkers the SWNT 2001 surface is activated using PBASE the biomarker is linked to the activated surface, and then the surface is passivated to form the passivated microelectrodes 2030 shown in (c). As an example, a 6 mM PBASE solution is prepared in DMF. The SWNT devices are then soaked in the PBASE solution for 1 hr to allow the functionalization of the SWNT by pi-pi interaction with the pyrene group followed by washing with ultrapure water. The biomarker (as described below) is then chemical linked to the SWNT/PBASE by incubating the device in the corresponding biomarker PBS solution for 1 hr followed by washing with phosphate buffered saline (PBS) and ultrapure water. After the biomarker is conjugation, the devices are washed and passivated using a solution of ethanolamine, Tween 20, and mercaptohexanol (MCH) followed by washing in PBS and water then are dried in air and stored at −20 C if not placed into use. Step (d) illustrates the addition of another layer 2032 to provide of site specific sampling wells 2034 and (e) illustrates the chip with antibodies 2036 attached to five of the microchips, a sixth microchip pairs of spring-loaded pin contacts 2044 without attached antibody serving as a reference cell 2138.

Matrix of Multiple Devices Functionalized with Different Biomarkers

Referring to FIG. 32C and FIG. 36, for detection using multiple biomarkers, a matrix 2040 of multiple pairs of microelectrodes 2018 is used. The multiple devices can be fabricated individually as described above using various different biomarkers, and then joined together on a substrate (either glass or plastic or Si wafer). Alternatively, a larger device with multiple electrode pairs can be provided directly during the chip dicing process. A similar SWNT deposition process can be performed on each electrode pair one at a time or a multi-channel deposition cell and multi-channel wave generator can be used. The biomarker functionalization process is carried out on the larger devices after the SWNT deposition process. A top mold 2042 that rests on the matrix 2040 holds multiple pairs of spring-loaded pin contacts 2044 which contact the two metal contacts 2012 of the microelectrode 2018. A wire 2046 provides a connection to the testing system.

### Data Measurement:

A reading using the sensor assembly described herein is taken by applying a linear voltage scan from −2V to 2V (or 0-1V) to the MED and recording the current across the pair of two electrodes using a semiconductor measurement unit, a potentiostat or any other electric devices that can simultaneously apply a voltage to the device while record the current. The current/voltage (I-V) measurement is taken during each intermediate step of the functionalization procedure described above to monitor the conjugation and passivation process with a baseline established before testing the sample.

To measure the response of the sensor device to the selected biomolecules, one drop of the sample solution (2˜4 uL) is added to the sensor's tip (referred to as the “finger electrode area”) and incubated for 10˜30 min followed by washing with PBS and ultrapure water and drying the sensor for 10 minutes.

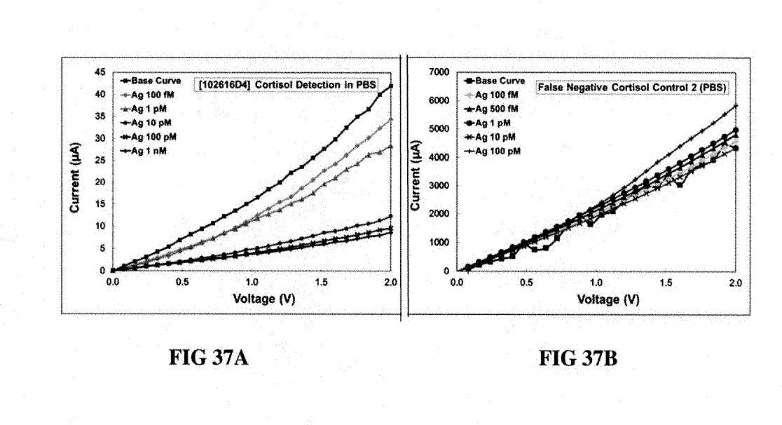

An example of an I-V curve for detection of cortisol Ab-Ag interaction and a base line, which provides a false negative control, is shown in FIGS. 37A and 37B. In the case of a fluctuating I-V data curve, to provide a stabilized data output and to avoid contaminants in the air, all measurements, including the baseline are taken with a drop of ultrapure water added on top of the finger electrode area of the sensor tip before every measurement was taken. If the device is pre-wired, the whole device can be immersed in ultrapure water while the measurement is being taken. A standard calibration curve, established using various samples of known concentrations, is used to determine the concentration of the unknown sample of interest. Alternatively, instead of the I-V curve, a direct measurement of current at a fixed voltage (0.5, 1, or 2V) or a direct measurement of resistance value can also be used to establish the correlation between the measured data and the concentration of the bio-molecule. Using a matrix of multiple devices such as shown in FIGS. 35-36, simultaneously readings or sequential readings can be achieved by using a setup that accommodates the device matrix.

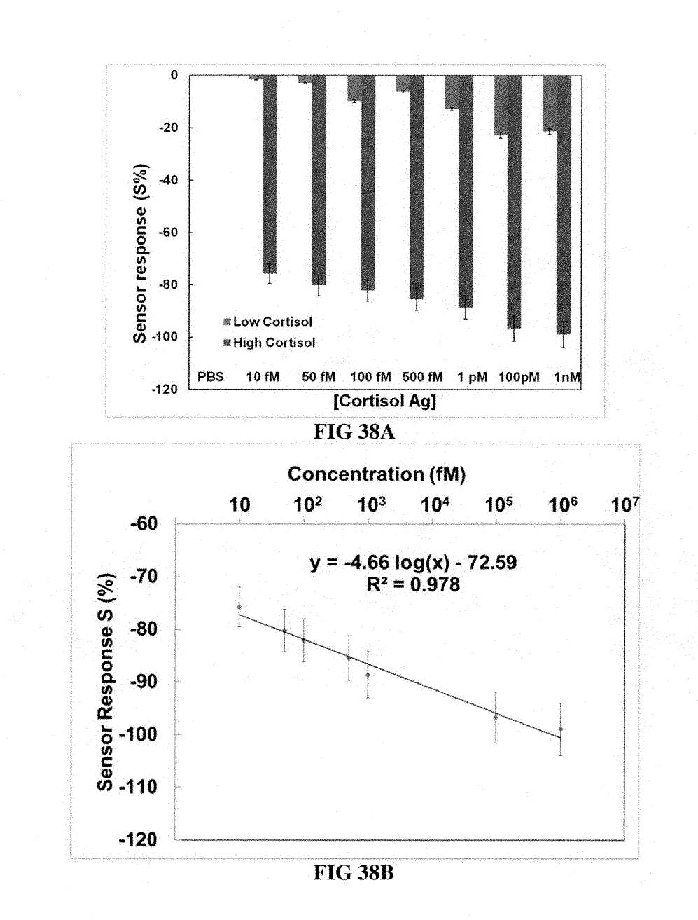

FIG. 38A is a bar graph showing the average response of multiple devices to varying concentrations of pooled cortisol compared to controls. The longer bars represent the response profile of the anti-cortisol Ab-conjugated devices to their corresponding (high) pooled salivary cortisol while the shorter bars represent the response of control devices to low pooled salivary cortisol exposed to the same dilution series of Ags. The X-axis is the concentrations of Ags in increasing order of exposure.

FIG. 38B is a calibration curve showing the average sensor response change as a function of increasing cortisol Ag. R² indicates the regression value for the linear trend line.

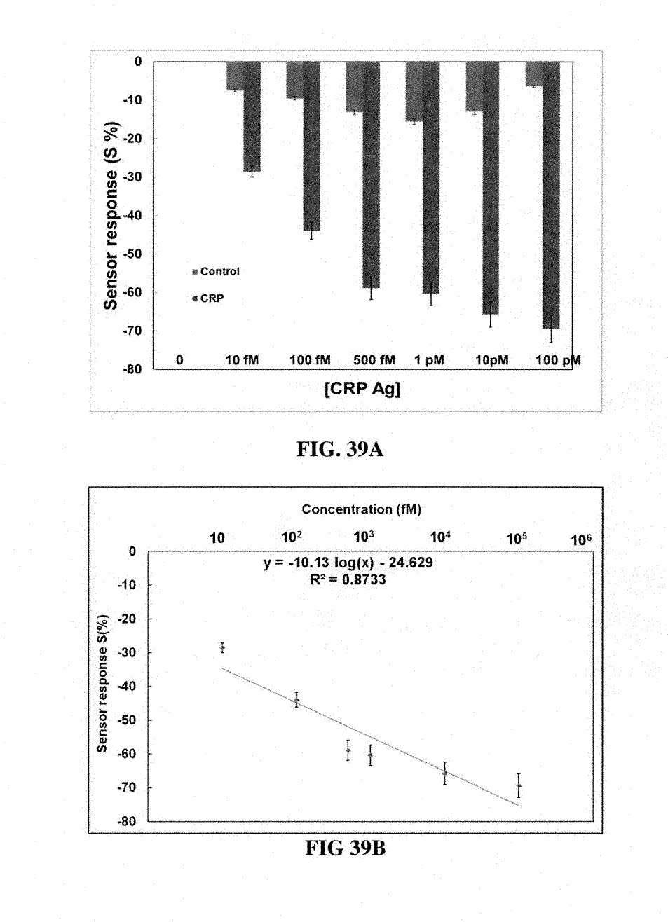

FIG. 39A, a bar graph derived from the calibration curve, summarizes the average negative response trend of multiple devices to varying concentrations of CRP when compared to controls. The long bars represent the response profile of the anti-CRP Ab-conjugated devices to its corresponding salivary CRP while the shorter (blue) bars represent the response of control devices exposed to the same dilution series.

FIG. 39B is a calibration curve showing the average sensor response change as a function of increasing CRP Ag. R² indicates the regression value for the trend line.

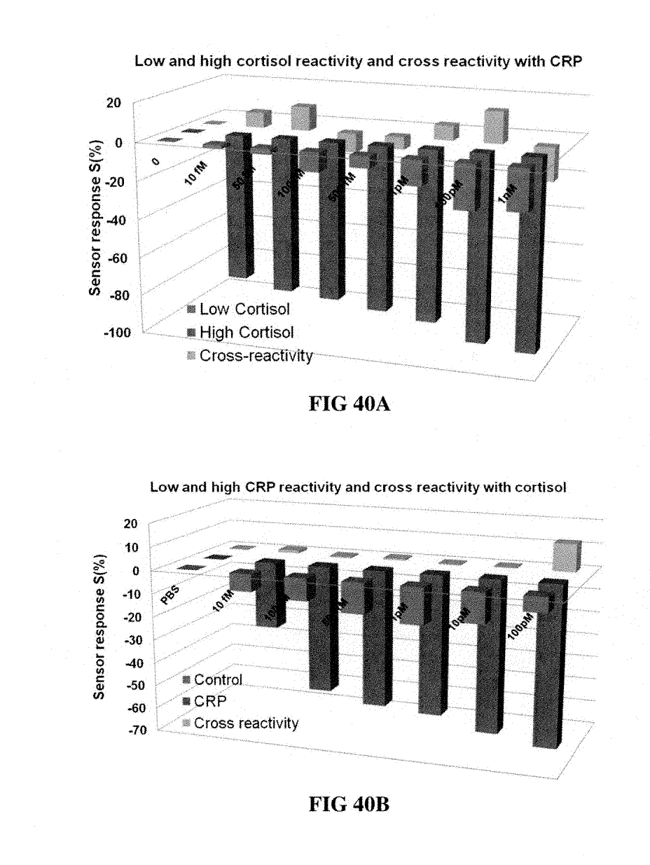

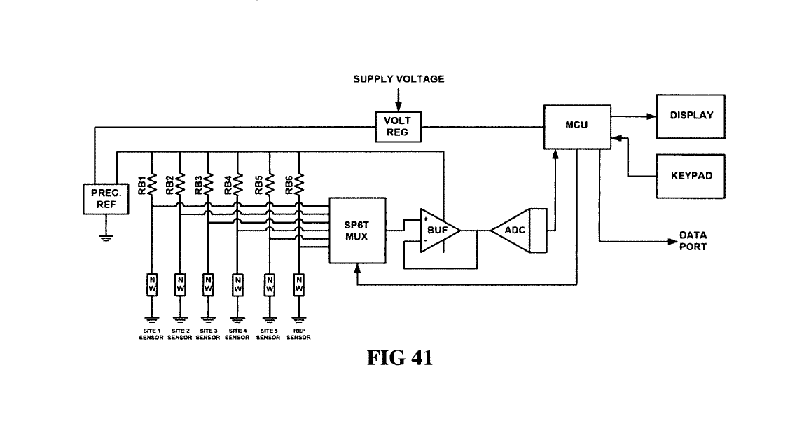

FIG. 40A is a three axis graph showing the average overall response of devices to varying concentrations of human salivary cortisol compared to controls (low salivary cortisols and cross reactivity with CRP Ags). The longer bars represent high cortisol, the shorter bars in the front represent low cortisol and the shortest bars in the rear show cross-reactivity.

FIG. 40B is a three axis graph showing the average overall response of devices to varying concentrations of human salivary CRP compared to controls (low salivary CRP and cross reactivity with cortisol Ags). The longer bars represent high cortisol, the shorter bars in the front represent low cortisol and the shortest bars to the rear show cross-reactivity.

### Biosample Pre-Treatment:

The biosamples evaluated were pretreated using devices, columns and cutoff membranes to eliminate extraneous materials which might interfere with the sensors.

The development of the electrical components and the availability of small components allowed construction of in vitro diagnostic (IVDs) devices with fast response times and low power requirements. Various matrices were tested to mitigate signal drift and provide a prototype constructed of durable, lightweight materials such as aluminum. Compact electronics including chip array interface connectors, a programmable complementary metal-oxide semiconductor (CMOS) multiplexing switch, a microcontroller display, and keypad interface are used to provide a portable prototype. A bridge-configuration circuit incorporating a variable gain differential amplifier (VGDA) facilitates controlling the overall output signal, mitigating drift and correcting for temperature and background matrix effect

### Biomarkers:

The device embodiments described herein are used as in vitro diagnostic devices to detect biomarkers or molecules which are indicators of various disease states such as liver cancer, colon cancer, or which can discriminate between infectious diseases, for example, detect and distinguish between dengue and chikungunya, between sepsis and multiple organ injury. The sensor described herein can be immersed in various bodily fluids which include, but are not limited to, exhaled breath, whole blood, plasma, serum, urine, semen, saliva, lymph fluid, meningeal fluid, amniotic fluid, glandular fluid, sputum, sweat, mucous, and cerebrospinal fluid. SWNT devices can be functionalized with antibodies, antigens, aptamers, affimers and microRNAs (miRNAs) and their variants for the detection of or related molecules from different matrices. The ability to simultaneously monitor several biomarkers instead of a single biomarker from a single sample in one assay is a major advantage that includes: (1) comprehensive diagnosis of any disease concomitantly reducing logistics burden and cost, (2) insight in to treatment strategy, which is the cornerstone of any successful therapy, especially in case of persistent sub-clinical infections or recurrence in case of cancer, (3) subsequent follow-up treatment, and (4) efficacy of drugs during clinical trials. Diseases and related biomarkers are listed Below.

### Detection of Hepatocellular Carcinoma (HCC):

Detection of HCC is accomplished using a panel of miRNAs. Because expression (upregulation/downregulation) of a single miRNA may not accurately differentiate HCC from other liver disease, the panel will include one or more chemically modified probes sensitive to miR-16, miR-18a, miR-21, miR-143, miR-199a, miR-215, miR-221, miR-222, miR-224, and miR-885 along with scrambled probes as negative controls. U6 snRNAs (RNU6A and 6B and miR-23b are used as reference controls. miR-16, 21, and 224 have diagnostic roles in HCC, miR-222 has a prognostic role, while miR-199a and 221 can play a dual role (diagnosis and prognosis). Serum miR-224 and miR-885 have been shown to be biomarkers of HCC at an early stage when compared to control patients.

### Colorectal Cancer (CRC):

Deregulation of specific miRNAs has been implicated in the development and progression of CRC. A CRC specific signature array with 6 miRNAs (miRNA-18a, miRNA-21, miRNA-29a, miRNA-92a, miRNA-141, miRNA-221) for IVDs are used for chemically modifying probe functionalized SWNTs to detect the six signature miRNAs in a single multiplexed assay on one platform.

### Multiorgan Injury:

Human saliva samples are used to determine the response of a panel of biomarkers, which include hTnT and CRP (heart and overall inflammation respectively), CYP1A1 (xenobiotic exposure), S100B (brain), and cortisol (overall stress) indicative of multi-organ injury in a single assay. A sample response bar graph and the corresponding calibration curve for the detection of cortisol with the devices is shown in FIGS. 38A and 38B. A sample response bar graph and the corresponding calibration curve for the detection of CRP with the devices are shown in FIGS. 39 A 39B. The response and cross reactivity of cortisol and CRP are shown in FIGS. 40A and 40B Proteu respectively.

### Sepsis:

The difficulty in using a single biomarker for diagnosing sepsis is in part due to the complex heterogeneity of the diseased state. Consequently, an array of biomarkers is needed. The sensor design supports a multiplex array format to quantitatively detect proteins and miRNAs (small non-coding RNA having ˜22 nucleotides) as biomarkers simultaneously in a single assay and platform from one blood sample of 20 μL. This provides a fast readout for determining accurate treatment options avoiding overuse and inappropriate therapy which is particularly critical in military field combat scenarios and in resource limited and austere environments. A combination of biomarkers reflecting the biology behind sepsis have been identified. Certain proteins, particularly Procalcitonin (PCT), C-reactive protein (CRP), TNF-related apoptosis-inducing ligand (TRAIL), and IL10, can accurately identify the presence of sepsis while increasing the sensitivity and specificity of diagnostic assays. This is evidenced by the biochemical changes that are characterized as the host response to infection.

Prior studies have indicated that PCT and CRP are proinflammatory biomarkers. PCT has been historically used in the diagnosis of sepsis and can distinguish between sepsis and SIRS resulting from sterile inflammation. Bacterial infection significantly increases PCT concentration in blood. CRP is an acute phase protein released by the liver after the onset of inflammation or tissue damage and frequently used to assess the presence of infection and sepsis and to differentiate bacterial and viral infections. TRAIL has been seen to be consistently up-regulated in virally infected patients and can complement bacterial induced host proteins such as PCT and CRP to distinguish between bacterial and viral infections. IL10 is a good biomarker for the anti-inflammatory cytokine response. Along with the proteins, miRNAs such as miR133a and miR486 are also used as sepsis biomarkers. miR133a has been reported to be elevated in sepsis and can predict mortality in critically ill patients. miR486 has also been reported to be elevated in sepsis versus healthy controls. A combination of protein and nucleotide biomarkers can successfully predict the underlying cause of sepsis and can provide earlier supportive treatment and constant monitoring. This will save substantial time and set the path towards accurate medical intervention.

### Cytomegalovirus and Herpesimplex Virus 1 and 2 Detection:

The Ags chosen for the detection of CMV are pp150 and pp52 synthetic peptides. CMV particles contain a basic phosphoprotein known as pp150 which has been shown to be reactive against anti-CMV human Ab. pp150 is highly immunogenic and is considered the primary antigenic candidate because it is recognized in nearly 100% of the seropositive subjects tested. pp52 is the nonstructural major DNA binding protein of CMV. This phosphoprotein is recognized by CMV IgM during the convalescent phase of a primary infection.

The Ags chosen for detection of HSV-1 and HSV-2 are glycoprotein G (gG1 and gG2) which are known to trigger a type specific response. Glycoproteins gB, gC, gD and gE trigger potent immune responses where some epitopes in these glycoproteins are shared between HSV-1 and HSV-2 resulting in significant degree of cross reactivity. However, no cross reactivity between glycoprotein gG1 and gG2 has been detected in HSV 1 and HSV-2, respectively. Hence, Abs to these glycoproteins are ideal for type-discriminating diagnostics. Before functionalizing the arrays with the pp150 and pp52, gG1 and gG2 antigens, the reactivity using Western blots is verified. Using all the above Ags on a single perform enable constructing a single assay to detect and differentiate between CMV, and HSV-1 and 2 in a hazardous setting.

### Dengue, (DENV; Flavivirus: Flaviviridae) and Chikungunya Viruses

CHIKV; Alphavirus; and Togaviridae are transmitted primarily by Aedes aegypti and Aedes albopictus mosquitoes. Humans are the primary host for the amplification of these diseases. The platform based on SWNTs described herein can detect and distinguish between DENY and CHIKV using a panel of antibodies (Abs) conjugated to the nanowire devices. Abs engineered against the non-structural protein 1 (NS1) protein of DENV will detect the four different serotypes of DENV. However, the results may vary between different DENV strains, viral infection status, and culture conditions. NS1 antigen detection is an option with good sensitivity and specificity in low resource settings where molecular tests are not a practical option. Additional Abs will include monoclonal Ab (MAbs) against DENV1 (D15-IF-3 DENV1) and DENV2 (3H5-DENV2). Anti-CHIKV Abs specific to native CHIKV lysate, will also be conjugated to the platform. These Abs detect the DENV and CHIKV on a single device using a rapid assay method with a simple read out.

### Multiplexed Sensor Arrays for Multiple Virus Detection on a Single Platform.

The sensor design comprises an array that can detect several viruses simultaneously. This feature (1) enables a single-step comprehensive diagnosis of mosquito-borne diseases with overlapping symptoms at an early stage, (2) provides insight into treatment efficacy, (3) allows evaluation of disease progression, (4) allows detection and distinguishing between all the etiological agents in one platform, and (5) provides the option to expand and build on this platform for multiple pathogen detection.

### Multiplexed Sensor Arrays for Accurate Determination of Lyme Disease (LD).

The unique and simple sensor design supports an array that will detect LD from a single serum sample without elaborate sample preparation. IgM Abs are produced early on in the disease, so detecting them can be helpful for identifying patients during the first few weeks of infection. Most methods may take several weeks following infection to detect IgGs. The detection time following infection can be greatly reduced using a highly sensitive detection device as described herein which has high sensitivity and thus reduces the Ab detection time following infection (i.e., at the early onset of the disease) over most prior available methods. Target selection on the nanowire site specific wells include engineered antigens (Ags) for the C6 peptide, a highly conserved, 25-amino acid immunogenic epitope derived from the variable major protein-like sequence, expressed (VlsE), protein of B. burgdorferi, outer surface protein A (OspA), OspB, OspC, OspD, Flagellin (Fla), and Borrelia membrane protein A (BmpA).

### Hand-Held/Table-Top Testing Prototype Electronics Design.

The electronic components for multi-channel data acquisition includes at least a high-precision reference source, multiplexer, high-resolution ADC, MCU, DSP engine, and LCD display. The electronic circuitry (FIG. 12) has several identical cells (6 are shown in the figure) in a disposable sensor array chip; five are for measuring the I-V curve of the samples and the 6^{th} measures the I-V curve for the reference control device. Measurements are made through a time-division multiplexer connected to a common ADC through a buffer amplifier controlled by the microcontroller. The use of a common ADC and buffer eliminates variability and improves the quality of the data acquisition.

The microcontroller will collect and average many ADC readings for each cell (i.e., samples and reference control chip) to provide an improved signal-to-noise ratio. The signals generated indicate the difference between the responses of the sample cells and the reference control cell and allow tracking of any changes due to temperature and time. Since the sensitivity of each sample cell is likely to vary, the gain and DC offset of the buffer amplifier can be varied by the microcontroller to optimize the dynamic range for each cell. This allows the circuit to use high gain to improve sensitivity and not risk saturating the ADC. The microcontroller is programmed with specific interpolation algorithms for each analyte based upon the responsivity of the sensor to concentration levels. This will allow the instrument to provide confident concentration analysis and display the results within the window of diagnostic limits.

### Hand-Held/Table-Top Testing Prototype Design.

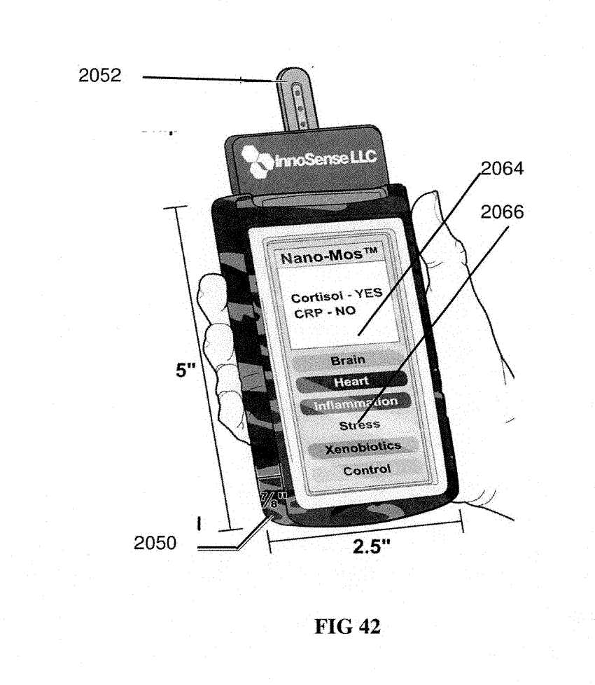

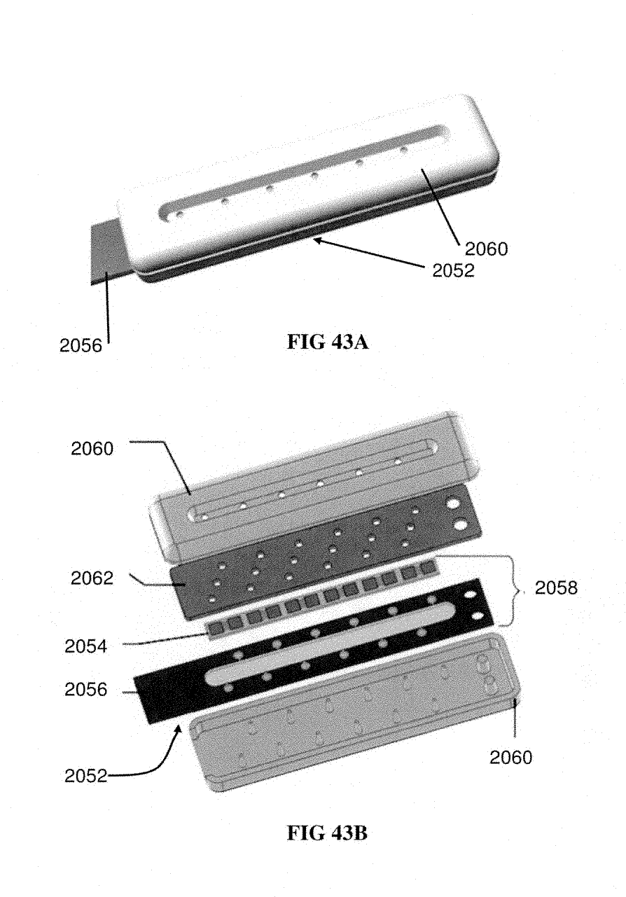

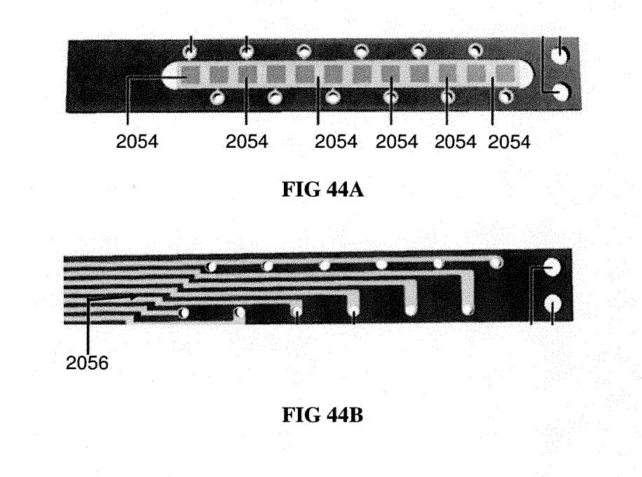

The handheld reader 2050 shown in FIG. 42 comprises 2 components that plug into the reader, namely the detector (not shown) and the disposable sensor package 2052. The detector, which is enclosed in the handheld reader 2050, uses commercial off-the shelf components for the USB C male connector and the 10 pin commercially available 0.8 mm pitch female connector. The electronic circuit is fabricated on a compact printed circuit board (PCB) that fits within the handheld reader 2050 enclosure is approximately 2.5″×1″×⅜″, as illustrated in FIG. 42. The disposable sensor package 2052 and the casing for the detector can be a 3D printed or injection molded using plastic. The disposable sensor chip 2058 comprises semiconductor wafers 2054 and circuit board 2056 as shown in FIG. 43B. FIGS. 44A and 44B show the front and rear surface of the assembled sensor chip 2058, with FIG. 44A showing multiple wafers sensors 2054 having different detection capabilities. The plastic enclosure 2060, which is spaced from the sensor chip 2058 by a gasket 2062 can be ultrasonically welded to seal the interior components from moisture. The detector has protective rubber plug and cap (not shown) to prevent dirt from contaminating the two external connectors when not in use, the plug and cap being permanently attached to the detector using a lanyard or living hinge. The viewing screen 2064 is made rugged to allow use as a portable device with rubber over wrap so it can operate in austere environments. The disposable sensor chip is provided as a separate unit in a sealed plastic bag. The sensor chip has a single adsorbent pad and individual transfer pads to allow liquid to be placed onto each sensor pad. The detector unit can be attached or integrated to the viewer as per DHA requirements and can be controlled via a user interface (keypad) 2066 on the viewer, while the sensor chip 2058 inserted into the detector will enable analyte measurements. This convenient design enables rapid sampling of saliva samples or any other biofluids such as serum, cerebrospinal fluid (CSF) or urine with a simple result readout.

Template-free, site-specific electrochemical approaches to the precise fabrication of individually addressable polymer nanowire or carbon nanotube microelectronic electrode junction devices have been demonstrated. A variety of different polymer nanowires or carbon nanotubes can be incorporated into an array format by electrochemically attaching to each individual junction a particular electroactive monomer. For example, a list of preferable nanomaterials for different sensors is set forth in Table 1 below:

TABLE 1

| Sensor | | Additional preferable |
|---|---|---|
| Type | Nanomaterials currently in use | nanomaterials |
| CO₂ Sensor | Amine functionalized polymer | Amine functionalized |
| | nanowires | carbon nanotubes |
| NO₂ and | Functionalized polyaniline | Modified single wall |
| MMH | nanowires | carbon nanotubes |
| Sensors | | |
| Alzheimer's | N-Hydroxy succinimide | N- Hydroxy succinimide |
| Disease | functionalized polymer | functionalized single wall |
| (AD) Sensor | nanowires followed by | carbon nanotubes followed |
| | conjugation with AD | by conjugation with AD |
| | proteins and biomarkers | proteins and biomarkers |

It is demonstrated herein that the excellent performance of the modular nanowire or nanotube microelectronic sensors in terms of their high sensitivity and their fast response for detecting toxic chemicals, gases and biomarkers are useful. These results demonstrate the versatility of modular nanowires or nanotubes microelectronic sensor technology for chemical and biological sensor applications.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

While various embodiments of the present invention have been described above, it should be understood that they have been presented by way of example only, and not of limitation. Likewise, the various diagrams may depict an example architectural or other configuration for the invention, which is done to aid in understanding the features and functionality that may be included in the invention. The invention is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to provide the desired features of the present invention. Also, a multitude of different constituent module names other than those depicted herein may be applied to the various components. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether flow control or other components, may be combined in a single package or separately maintained and may further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An improved method for producing a sensor comprising
forming a first and a second noble metal electrode on a silicon substrate, said electrodes separated by a gap of 0.5 to 4.0 μm, said electrodes connected to a power source and means for measuring current and/or voltage between the first and second noble metal electrodes,
wherein the improvement comprises forming a nano-network of functionalized carbon nanowires or nanotubes (SWNTs) in situ, the network of nanotubes spanning the gap and providing an electrically conductive pathway connecting the first and second noble metal electrodes
said SWNTs functionalized by
a) treating the surface of the SWNT with an activating agent
b) electrochemically depositing and binding disease detecting agents or biomarkers on the activated SWNT surfaces to form, functionalized surfaces, and
c) then passivating the functionalized surfaces of the SWNTs,
said disease detecting agents being specific to provide diagnosis and/or prognosis of a target disease.

2. The improved method of claim 1 wherein the SWNT is activated by soaking in a DMF solution of 1-pyrenebutanoic acid succinimidyl ester (PBASE).

3. The improved method of claim 1 wherein the target disease detecting agent is selected from the group consisting of antibodies, antigens, aptamers, affirmers, microRNAs and variants thereof.

4. The improved method of claim 3 wherein the target disease for detection or prognosis is hepatocellular carcinoma and the disease detecting agents comprises one or more microRNAs.

5. The improved method of claim 3 wherein the target disease for detection or prognosis is colorectal cancer and the disease detecting agents comprises one or more microRNAs.

6. The improved method of claim 3 wherein the target disease for detection or prognosis is multiorgan injury and the disease detecting agents comprises a panel comprising one or more of of hTnT, CRP, CYP1A1 and cortisol and S100B.

7. The improved method of claim 3 wherein the target disease for detection or prognosis is sepsis and the disease detecting agents comprise one or more of procalcitonin (PCT), C-reactive protein (CRP), TNF-related apoptosis-inducing ligand (TRAIL), miRNA 133a and miRNA 486.

8. The improved method of claim 3 wherein the target disease for detection or prognosis is cytomegalovirus pp150 or pp52 synthetic peptides.

9. The improved method of claim 3 wherein the target disease for detection or prognosis is Herpesimplexvirus 1 and 2 and the disease detecting agents comprises one or more of glycoprotein G (gG1 and gG2.

10. The improved method of claim 3 wherein the target diseases for detection or prognosis are dengue and chikungunya and the disease detecting agents comprise one or more of NS1 antigen detectors, monoclonal Ab and anti-CHIKV antibodies.

11. The improved method of claim 3 wherein the target diseases for detection or prognosis is lyme disease and the disease detecting agents comprise one or more of engineered antigens of C6 peptide.

12. A sensor array comprising multiple discrete sensors, each of the multiple discrete sensors comprising a first and a second noble metal electrode on a silicon substrate, said electrodes separated by a gap of 0.5 to 4.0 the first and second electrodes in electrically communication via a nano-network of functionalized polymer nanowires or carbon nanotubes (SWNTs) spanning the gap and providing the electrically conductive pathway connecting the first and second noble metal electrodes,
the multiple discrete sensors comprising a reference cell and multiple detection sensors functionalized to detect a disease or biological function.

13. The sensor array of claim 12 wherein the detection sensors are each functionalized to detect a different disease or biological function.

14. The sensor array of claim 13 wherein the detection sensors are functionalized to detect one or more of cancers, multiorgan injury, sepsis, viruses, lyme disease, dengue and chikungunya.
